(19) United States
(12) Patent Application Publication
(10) Pub. No.: US 2009/0074698 A1
(43) Pub. Date: Mar. 19, 2009

(54) COSMETIC COMPOSITIONS CONTAINING AT LEAST ONE CATIONIC COPOLYMER, AT LEAST ONE CYCLODEXTRIN AND AT LEAST ONE SURFACTANT, AND USES THEREOF

(76) Inventor: Olga BIGANSKA, Asnieres sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/210,572

(22) Filed: Sep. 15, 2008

Related U.S. Application Data
(60) Provisional application No. 60/960,286, filed on Sep. 24, 2007.

(30) Foreign Application Priority Data
Sep. 14, 2007 (FR) 07/57581

(51) Int. Cl. A61K 8/81; A61K 8/88; A61Q 5/12

(52) U.S. Cl. 424/70.16; 424/70.17

(57) ABSTRACT

The disclosure relates to novel cosmetic compositions comprising, in an aqueous medium:
(i) at least one cationic polymer, which is obtained by polymerization of a monomer mixture comprising:
   a) at least one vinyl monomer substituted with at least one amino group,
   b) at least one hydrophobic nonionic vinyl monomer chosen from those of formulae (I) and (II):

CH₂═C(X)Z, (I)

CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃ and —(CH₂)ₓSi(R)₃;
x is an integer ranging from 1 to 6;
each R independently is a C₁-C₃₀ alkyl group; and
each R1 independently is chosen from a C₁-C₃₀ alkyl group, a hydroxylated C₂-C₃₀ alkyl group, and a halogenated C₁-C₃₀ alkyl group;
   c) at least one associative vinyl monomer,
   d) at least one hydroxylated nonionic vinyl monomer, and
   e) one or more semi-hydrophobic vinyl surfactant monomers,
(ii) at least one cyclodextrin or a derivative thereof and
(iii) at least one surfactant.

The compositions according to the present disclosure may be used as rinse-out products especially for washing and/or conditioning keratin materials.

This application claims benefit of U.S. Provisional Application No. 60/960,286, filed Sep. 24, 2007, the contents of which are incorporated herein by reference. This application also claims benefit of priority under 35 U.S.C. § 119 to French Patent Application No. FR 0757581, filed Sep. 14, 2007, the contents of which are also incorporated herein by reference.

The present disclosure relates to compositions comprising, in a physiologically acceptable aqueous medium, at least one cyclodextrin, at least one surfactant and at least one particular cationic copolymer. The disclosure also relates to compositions also comprising at least one conditioning agent. The disclosure also relates to the use of the particular at least one cationic copolymer as an agent for suspending the complex formed from the at least one cyclodextrin and the at least one surfactant.

It is well known that hair which has been sensitized (i.e. damaged and/or embrittled) to varying degrees under the action of atmospheric agents or under the action of mechanical or chemical treatments, such as dyes, bleaches and/or permanent-waving, is often difficult to disentangle and to style, and lacks softness.

It has already been recommended to use conditioners, in particular insoluble conditioners, in compositions for washing or caring for keratin material such as the hair, in order to disentangle the hair and to give it softness, sheen and suppleness.

Given the insoluble nature of certain conditioning agents, for instance silicones or oils, it is sought to maintain the conditioning agents in uniform dispersion in the medium without causing a reduction in the viscosity and the detergent and foaming properties of the compositions. The conditioning agents, especially silicones or oils, should also be conveyed onto the treated keratin materials so as to give them, following application, softness, sheen and disentangling properties.

It is also known that products, in particular cosmetic products, having an iridescent, moiré or metallized appearance are widely appreciated by consumers for their aesthetic appearance and for the rich appearance of the product. The agents that give this effect are pearlescent or nacreous agents generally comprising crystals that remain dispersed in the compositions and that reflect light.

Long-chain ester derivatives are widely used for making compositions, especially cosmetic compositions, nacreous. However, these derivatives may have crystallization problems leading to a change in the viscosity of the compositions over time.

Long-chain ether or thioether derivatives such as those described in patent applications EP 457 688 and WO 98/03155 are also known. However, these derivatives opacify the compositions without giving them any or sufficient nacreous effect.

It has been found that, due to the low density of these nacreous agents, they often have the drawback of rising to the surface of the shampoo and of forming thereon a layer that consumers find unaesthetic.

Furthermore, in certain cases, these fatty-chain compounds have the drawback of giving the hair a laden feel and lack of lightness and volume.

There is thus still a need for novel nacreous agents that do not have the drawbacks mentioned above and that also allow the use of insoluble conditioning agents and in particular silicones.

Cyclodextrins have been used for making compositions nacreous, especially shampoos: see especially EP 1 499 280. However, the compositions are not sufficiently stable upon storage, in particular at high temperatures (about 45° C.), and the nacreous effect may disappear over time.

To thicken and stabilize cosmetic compositions containing insoluble conditioning agents, stabilizers such as crosslinked acrylic polymers of the Carbopol 980 type are frequently used. However, these stabilizers have the drawback of reducing the cosmetic performance qualities of shampoos, in particular by making the hair more laden and coarser. Furthermore, the compositions are still not sufficiently stable upon storage, in particular at high temperatures (about 45° C.). The nacreous effect disappears over time.

EP1779840 discloses compositions comprising surfactants, cyclodextrin and acrylic polymer. The acrylic polymers are different from cationic copolymers according to the present disclosure.

The Applicant has discovered that it is possible to formulate compositions, especially cosmetic compositions for treating keratin materials, and more particularly shampoos with a nacreous appearance, while at the same time having the desired aesthetic and cosmetic properties, by using in these compositions at least one cyclodextrin, at least one surfactant, and at least one particular cationic polymer.

One subject of the disclosure is compositions, especially cosmetic compositions, comprising, in an aqueous medium, especially a physiologically acceptable medium, and more particularly a cosmetically acceptable medium:

(i)—at least one cationic polymer, which is a product of polymerization of a monomer mixture comprising:
a) at least one vinyl monomer substituted with at least one amino group,
b) at least one hydrophobic nonionic vinyl monomer chosen from those of formulae (I) and (II):

CH₂═C(X)Z, and (I)

CH₂═CH—OC(O)R; (II)

in which formulae (I) and (II):

X represents H or a methyl group;

Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃—C(O)NH(CH₂)ₓSi(R)₃ and —(CH₂)ₓSi(R)₃;

x represents an integer ranging from 1 to 6; and

each R independently represents a C₁-C₃₀ alkyl group;

and
c) at least one associative vinyl monomer,
d) at least one hydroxylated nonionic vinyl monomer, and
e) one or more semi-hydrophobic vinyl surfactant monomers,

(ii)—at least one cyclodextrin or a derivative thereof, and

(iii)—at least one surfactant.

The compositions according to the disclosure have the advantage of spreading easily and of being uniformly distributed along the keratin fibres.

The presence of this at least one copolymer may not reduce the cosmetic properties of the compositions, and may even improve them, especially as regards the suppleness, smoothness, sheen, volume and/or manageability.

The compositions have very good homogeneity and good stability of the nacreous effect, and also a satisfactory viscosity for application to keratin materials. There may be no phase separation or any disappearance of the nacreous effect over time.

In particular, there may be no uncontrolled thickening or leaching of the composition over time. Finally, the compositions have a non-runny, fondant texture. The foam rinses out easily.

A subject of the present disclosure is also the use of at least one cationic polymer (i) as an agent for suspending the insoluble complex formed by the at least one cyclodextrin and at least one surfactant in a cosmetic composition, in particular a washing and/or conditioning composition, comprising at least one physiologically acceptable aqueous medium comprising at least one cyclodextrin and at least one surfactant.

Another subject of the present disclosure is a washing and/or conditioning process using such compositions.

Another subject of the present disclosure concerns the use of at least one cationic polymer (i) as described below, in, or for the manufacture of, a cosmetic composition comprising at least one cyclodextrin and at least one surfactant.

Other subjects of the disclosure will become apparent on reading the description and the examples that follow.

According to the present disclosure, the term “keratin materials” means the hair, the eyelashes, the eyebrows, the skin, the nails, mucous membranes or the scalp.

The terms “pearlescent agent” and “nacreous agent” mean an agent that produces a nacreous, iridescent, moiré or metallized appearance or effect.

One of the characteristics of the present disclosure is the presence of at least one cationic polymer that is obtained by polymerization of a monomer mixture comprising a) at least one vinyl monomer substituted with at least one amino group, b) at least one hydrophobic nonionic vinyl monomer, c) at least one associative vinyl monomer and d) at least one hydroxylated nonionic vinyl monomer. In one aspect of the present disclosure, the monomers constituting the at least one cationic copolymer also comprise e) at least one semi-hydrophobic vinyl surfactant monomer. The monomers a) to e) are different from each other.

In at least one aspect of the disclosure, the at least one cationic polymer (i) is a thickening polymer.

For the purposes of the present disclosure, the term “thickening polymer” means a polymer that, when introduced at 1% by weight into an aqueous or aqueous-alcoholic solution containing 30% by weight of ethanol, and at pH 7, makes it possible to achieve a viscosity of at least 100 cps at 25° C. and at a shear rate of 1 s⁻¹. This viscosity may be measured using a viscometer with cone-plate geometry, for example a Haake RS 600 rheometer. Preferably, these polymers make it possible to increase the viscosity of the compositions in which they are present by at least 50 cps at 25° C. and at 1 s⁻¹.

The at least one cationic polymer (i) used in the composition according to the disclosure, and the process for manufacturing it, are especially described in international patent application WO 2004/024 779.

For the purposes of the present disclosure, the term “vinyl monomer” means a monomer comprising one or more groups R₀CH═C(R₀)—, in which each R₀ is independently H, a C₁-C₃₀ alkyl, —C(O)OH or C(O)OR₀′, —O—C(O)OR₀′, —C(O)NHR₀′, —C(O)NHR₀′ or C(O)NR₀′R₀″. R₀′ and R₀″, which may be identical or different, being a C₁-C₃₀ alkyl group.

Thus, for example, for the purposes of the present disclosure, (meth)acrylates and (meth)acrylamides are vinyl monomers.

As explained previously, the monomer mixture for preparing the at least one cationic polymer (i) used in the composition according to the present disclosure comprises at least one vinyl monomer substituted with at least one amino group.

The vinyl monomers substituted with at least one amino group that may be used for the preparation of the cationic polymer used according to the present disclosure are basic, polymerizable, ethylenically unsaturated monomers. The amine groups may be derived from monoamino, diamino or polyamino alkyl groups, or from heteroaromatic groups comprising a nitrogen atom. The amine groups may be primary, secondary or tertiary amines. These monomers may be used in the form of an amine or in the form of a salt.

In one aspect of the disclosure, at least one vinyl monomer substituted with at least one or more amine group is chosen from, but not limited to:

mono(C₁-C₄)alkylamino(C₁-C₈)alkyl (meth)acrylates,

di(C₁-C₄)alkylamino(C₁-C₈)alkyl (meth)acrylates, preferably di(C₁-C₄)alkylamino(C₁-C₆)alkyl (meth)acrylates,

mono(C₁-C₄)alkylamino(C₁-C₈)alkyl(meth)acrylamides,

di(C₁-C₄)alkylamino(C₁-C₈)alkyl(meth)acrylamides,

(meth)acrylamides with heterocyclic group(s) containing a nitrogen atom,

(meth)acrylates with heterocyclic group(s) containing a nitrogen atom, and

nitrogenous heterocycles containing vinyl group(s).

The at least one vinyl monomer substituted with at least one amino group that may further include:

mono- or di(C₁-C₄ alkyl)amino(C₁-C₄ alkyl) (meth)acrylates, such as 2-(N,N-dimethylamino)ethyl (meth)acrylate, 3-(N,N-dimethylamino)propyl (meth)acrylate, 4-(N,N-dimethylamino)butyl (meth)acrylate, (N,N-dimethylamino)-t-butyl (meth)acrylate, 2-(N,N-diethylamino)ethyl (meth)acrylate, 3-(N,N-diethylamino)propyl (meth)acrylate, 4-(N,N-diethylamino)butyl (meth)acrylate, 2-(N,N-dipropylamino)ethyl (meth)acrylate, 3-(N,N-dipropylamino)propyl (meth)acrylate and 4-(N,N-dipropylamino)butyl (meth)acrylate;

mono- or di(C₁-C₄ alkyl)amino(C₁-C₄ alkyl)(meth)acrylamides such as N′-(2-N,N-dimethylamino)ethyl(meth)acrylamide and N′-(3-N,N-dimethylamino)propylacrylamide;

(meth)acrylamides or (meth)acrylates with a heterocyclic group comprising a nitrogen atom, such as N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)-methacrylamide and N-(4-morpholinyl)acrylamide; and

nitrogenous heterocycles containing vinyl group(s), such as 2-vinylpyridine and 4-vinylpyridine.

When the monomers are in the form of salts, they may be mineral salts, such as hydrochloride, sulfate and phosphate salts; or alternatively organic acid salts, such as acetate, maleate and fumarate salts.

Additional examples of the at least one vinyl monomer substituted with at least one amino group include:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate.

The at least one vinyl monomer substituted with at least one amino group generally represents from 10% to 70% by weight, from 20% to 60% by weight, and even from 30% to 40% by weight relative to the total weight of the monomer mixture.

As explained previously, the monomer mixture for preparing the at least one cationic polymer (i) used according to the present disclosure also comprises at least one hydrophobic nonionic vinyl monomer b).

The at least one hydrophobic nonionic vinyl monomer for the preparation of the at least one cationic polymer used according to the present disclosure is generally chosen from compounds of the formulae (I) and (II):

CH₂═C(X)Z, (I)

CH₂═CH—OC(O)R; (II)

in which, in each of the formulae (I) and (II):

X represents H or a methyl group;

Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃ and —(CH₂)ₓSi(R)₃;

x represents an integer ranging from 1 to 6;

each R independently represents a C₁-C₃₀ alkyl group; and

each R¹ independently represents a C₁-C₃₀ alkyl group, a C₂-C₃₀ hydroxyalkyl group or a C₁-C₃₀ haloalkyl group.

Nonlimiting examples include: C₁-C₃₀ alkyl (meth)acrylates; (C₁-C₃₀ alkyl)(meth)acrylamides; styrene, substituted styrenes and in particular vinyltoluene (or 2-methylstyrene), butylstyrene, isopropylstyrene and para-chlorostyrene; vinyl esters and in particular vinyl acetate, vinyl butyrate, vinyl caprylate, vinyl pidolate and vinyl neodecanoate; unsaturated nitriles and in particular (meth)acrylonitrile and acrylonitrile; and unsaturated silanes and in particular trimethylvinylsilane, dimethylethylvinylsilane, allyldimethylphenylsilane, allyltrimethylsilane, 3-acrylamidopropyltrimethylsilane and 3-trimethylsilyipropyl methacrylate.

The at least one hydrophobic nonionic vinyl monomer is further chosen from C₁-C₃₀ alkyl acrylates and C₁-C₃₀ alkyl methacrylates, such as ethyl acrylate, methyl methacrylate and 3,3,5-trimethylcyclohexyl methacrylate.

The at least one hydrophobic nonionic vinyl monomer generally represents from 20% to 80% by weight, from 20% to 70% by weight, and even from 50% to 65% by weight relative to the total weight of the monomer mixture.

The at least one associative vinyl monomer that may be used for the preparation of the at least one cationic polymer (i) used according to the present disclosure is generally chosen from compounds having an ethylenically unsaturated end (i)′ for addition polymerization with other monomers of the system; a polyoxyalkylene central portion (ii)′ for giving the polymers selective hydrophilic properties, and a hydrophobic end (iii)′ for giving the polymers selective hydrophobic properties.

In one aspect of the disclosure, the ethylenically unsaturated end (i)′ of the associative vinyl monomer(s) may be derived from an α,β-ethylenically unsaturated monocarboxylic or dicarboxylic acid or anhydride, including a C₃ or C₄ monocarboxylic or dicarboxylic acid or anhydride. Alternatively, the end (i)′ of the associative monomer may be derived from an allyl ether or a vinyl ether; from a nonionic urethane monomer substituted with a vinyl group, as disclosed in the reissued US patent Re. 33,156 or in U.S. Pat. No. 5,294,692; or a product of reaction of urea substituted with a vinyl group, as disclosed in U.S. Pat. No. 5,011,978.

The central portion (ii)′ of the at least one associative vinyl monomer(s) may be a polyoxyalkylene segment comprising 5 to 250, from 10 to 120 and even from 15 to 60 C₂-C₇ alkylene oxide units. Central portions (ii)′ include, but are not limited to, polyoxyethylene, polyoxypropylene and polyoxybutylene segments comprising 5 to 150, 10 to 100 and even 15 to 60 ethylene oxide, propylene oxide or butylene oxide units, and random or non-random blocks of ethylene oxide, propylene oxide or butylene oxide units. In one embodiment of the disclosure, the central portions are polyoxyethylene segments.

The hydrophobic end (iii)′ of the at least one associative monomer(s) may be a hydrocarbon-based fragment belonging to one of the following hydrocarbon classes: a linear alkyl, a C₂-C₄₀ alkyl substituted with an aryl group, a phenyl substituted with a C₂-C₄₀ alkyl group, a branched alkyl, a alicyclic group, and a complex ester.

For the purposes of the present disclosure, the term “complex ester” means any ester other than a simple ester.

For the purposes of the present disclosure, the term “simple ester” means any ester of an unsubstituted, linear or branched saturated C₁-C₃₀ aliphatic alcohol.

Non-limiting examples of hydrophobic ends (iii)′ of the at least one associative monomer(s) are linear or branched alkyl groups of 8 to 40 carbon atoms, such as capryl (C₈), isooctyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀) and lacceryl (C₃₂) groups.

Non-limiting examples of linear or branched alkyl groups containing 8 to 40 carbon atoms and derived from a natural source include alkyl groups derived from hydrogenated groundnut oil, soybean oil and canola oil (predominantly C₁₈), C₁₆-C₁₈ hydrogenated tallow oil; and C₁₀-C₃₀ hydrogenated terpenols, such as hydrogenated geraniol (branched C₁₀), hydrogenated farnesol (branched C₁₅) and hydrogenated phytol (branched C₂₀).

Non-limiting examples of phenyls substituted with a C₂-C₄₀ alkyl include octylphenyl, nonylphenyl, decylphenyl, dodecylphenyl, hexadecylphenyl, octadecylphenyl, isooctylphenyl and sec-butylphenyl.

Non-limiting examples of C₈-C₄₀ alicyclic groups include groups derived from sterols of animal origin, such as cholesterol, lanosterol and 7-dehydrocholesterol; or alternatively derivatives of plant origin, such as phytosterol, stigmasterol or campesterol; or alternatively derivatives obtained from microorganisms, such as ergosterol or mycrosterol. Other C₈-C₄₀ alicyclics that may be used in the present disclosure are, for example, cyclooctyl, cyclododecyl, adamantyl and decahydronaphthyl, and groups derived from natural C₈-C₄₀ alicyclics compounds such as pinene, hydrogenated retinol, camphor and isobornyl alcohol.

The C₂-C₄₀ alkyl groups substituted with an aryl group may be, for example, 2-phenylethyl, 2,4-diphenybutyl, 2,4,6-triphenylhexyl, 4-phenylbutyl, 2-methyl-2-phenylethyl or 2,4,6-tris(1′-phenylethyl)phenyl.

Examples of C₈-C₄₀ complex esters that may be used as end (iii), include, but are not limited to, hydrogenated castor oil (mainly 12-hydroxystearic acid triglyceride); 1,2-diacyl glycerols such as 1,2-distearyl glycerol, 1,2-dipalmitol glycerol and 1,2-dimyristyl glycerol; di-, tri- or polyesters of sugars, such as 3,4,6-tristearyl glucose or 2,3-dilauryl fructose; and sorbitan esters such as those disclosed in U.S. Pat. No. 4,600,761.

The at least one associative vinyl monomer that may be used according to the present disclosure may be prepared via any method known in the prior art. Reference may be made, for example, to U.S. Pat. No. 4,421,902; U.S. Pat. No. 4,384,096; U.S. Pat. No. 4,514,552; U.S. Pat. No. 4,600,761; U.S. Pat. No. 4,616,074; U.S. Pat. No. 5,294,692; U.S. Pat. No. 5,292,843; U.S. Pat. No. 5,770,760 and U.S. Pat. No. 5,412,142.

In one aspect of the invention, the at least one associative vinyl monomer c) that may be used according to the present disclosure is chosen from the compounds of formula (III):

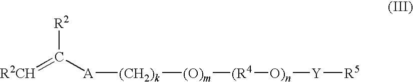

in which:

each R² is independently H, a methyl group, a group

—C(O)OH or a group C(O)OR³;

R³ is a C₁-C₃₀ alkyl;

A is a group —CH₂C(O)O—, —C(O)O—, —O—, CH₂O, —NHC(O)NH—, —C(O)NH—, —Ar—(CE₂)_{z}—NHC(O)O—, —Ar—(CE₂)_{z}—NHC(O)NH— or —CH₂CH₂—NHC(O)—;

Ar is a divalent aryl group;

E is H or a methyl group;

z is equal to 0 or 1;

k is an integer ranging from 0 to 30;

m is equal to 0 or 1, on condition that when k=0, m=0, and when k ranges from 1 to 30, m is equal to 1;

(R⁴—O)ₙ is a polyoxyalkylene, which is a homopolymer, a random copolymer or a block copolymer, with C₂-C₄ oxyalkylene units,

R⁴ is C₂H₄, C₃H₆, C₄H₈, or mixtures thereof,

n is an integer ranging from 5 to 250,

Y is —R⁴O—, —R⁴NH—, —C(O)—, —C(O)NH—, R⁴NHC(O)NH— or —C(O)NHC(O)—;

R⁵ is a substituted or unsubstituted alkyl chosen from linear C₈-C₄₀ alkyls, branched C₈-C₄₀ alkyls, C₈-C₄₀ alicyclics, phenyls substituted with a C₂-C₄₀ alkyl group, C₂-C₄₀ alkyls substituted with an aryl group, and C₈-C₈₀ complex esters,

the alkyl group R⁵ optionally comprising one or more substituents chosen from hydroxyl, alkoxy and halo groups.

In one aspect of the disclosure, the at least one associative vinyl monomer is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, polyethoxylated cholesteryl (meth)acrylates, and mixtures thereof, in which the polyethoxylated portion of the monomer comprises from 5 to 100, from 10 to 80 and even from 15 to 60 ethylene oxide units.

In at least one aspect of the disclosure, the at least one associative vinyl monomer is chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates and polyethoxylated lauryl (meth)acrylates, in which the polyethoxylated portion of the monomer comprises from 10 to 80, from 15 to 60, and even from 20 to 40 ethylene oxide units.

In one aspect of the disclosure, the at least one associative vinyl monomer represents from 0.001% to 25% by weight, preferably from 0.01% to 15% by weight, and even from 0.1% to 10% by weight of the monomer mixture.

The at least one semi-hydrophobic vinyl surfactant monomer optionally present in the monomer mixture can moderate the associative properties of the at least one cationic associative polymer that contains them, thus producing aqueous gels having a very good texture and very good rheological properties.

For the purposes of the present disclosure, the term “semi-hydrophobic vinyl surfactant monomer” means a monomer with a structure similar to that of an associative monomer, but which has a substantially non-hydrophobic end and thus does not give the polymers associative properties.

The associative property of a polymer is linked to the property in a given medium, of the molecules of the said polymer to associate with each other, or to associate with molecules of a co-agent, in general a surfactant, which is reflected in a certain concentration range by an increase in the viscosity of the medium.

The at least one semi-hydrophobic vinyl surfactant monomer is generally a compound having two parts:

(i) an unsaturated end group to allow addition polymerization with the other monomers of the reaction mixture, and

(ii) a polyoxyalkylene group to attenuate the associations between the hydrophobic groups of the polymer or the hydrophobic groups of the other materials that may be present in the composition comprising the polymer.

The end providing the vinyl or ethylenic unsaturation for the addition polymerization is preferably derived from an α,β-ethylenically unsaturated monocarboxylic or dicarboxylic acid or anhydride, particularly a C₃-C₄ monocarboxylic or dicarboxylic acid, or an anhydride of this acid. Alternatively, the end A may be derived from an allylic ether, a vinyl ether or a nonionic unsaturated urethane.

The polymerizable unsaturated end A may also be derived from a C₈-C₃₀ unsaturated fatty acid comprising one or more free carboxyl functional groups. This C₈-C₃₀ group forms part of the unsaturated end A and is different from the pendent hydrophobic groups of the associative monomers, which are separated from the unsaturated end of the associative monomer by a hydrophilic spacer group.

The polyoxyalkylene portion B comprises a long-chain polyoxyalkylene segment, which is essentially similar to the hydrophilic portion of the associative monomers. In one aspect of the disclosure, polyoxyalkylene portions B include C₂-C₄ polyoxyethylene, polyoxypropylene and polyoxybutylene units comprising from 5 to 250 and even from 10 to 100 oxyalkylene units. When the semi-hydrophobic vinyl surfactant monomer comprises more than one type of oxyalkylene unit, these units may be distributed randomly, non-randomly or in blocks.

In one aspect of the disclosure, the semi-hydrophobic vinyl surfactant monomer(s) is (are) chosen from the compounds of formula (IV) or (V):

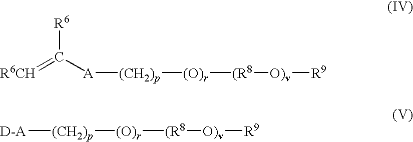

in which, in each formula (IV) or (V),

each R⁶ independently represents H, a C₁-C₃₀ alkyl, —C(O)OH or C(O)OR⁷;

R⁷ is a C₁-C₃₀ alkyl;

A is a group —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}—NHC(O)NH— or —CH₂CH₂NHC(O)—;

Ar is a divalent aryl group;

E is H or a methyl group;

z is equal to 0 or 1;

p is an integer ranging from 0 to 30;

r is equal to 0 or 1, on condition that when p is equal to 0, r is equal to 0, and when p ranges from 1 to 30, r is equal to 1,

(R₈—O)ᵥ is a polyoxyalkylene which is a homopolymer, a random copolymer or a block copolymer with C₂-C₄ oxyalkylene units, in which R⁸ is C₂H₄, C₃H₆, C₄H₈ or mixtures thereof, and v is an integer ranging from 5 to 250;

R⁹ is H or a C₁-C₄ alkyl; and

D is an unsaturated C₈-C₃₀ alkenyl or an unsaturated C₈-C₃₀ alkyl substituted with a carboxyl group.

In one embodiment of the disclosure, the monomer mixture comprises a semi-hydrophobic vinyl surfactant monomer having one of the following formulae:

CH₂═CH—O(CH₂)ₐO(C₃H₆O)_{b}(C₂H₄O)_{c}H or

CH₂═CHCH₂O(C₃H₆O)_{d}(C₂H₄O)ₑH;

in which:

a is equal to 2, 3 or 4;

b is an integer ranging from 1 to 10;

c is an integer ranging from 5 to 50;

d is an integer ranging from 1 to 10; and

e is an integer ranging from 5 to 50.

Non-limiting examples of the at least one semi-hydrophobic vinyl surfactant monomers are the polymerizable emulsifiers sold under the references Emulsogen® R109, R208, R307, RAL109, RAL208 and RAL307 by the company Clariant; BX-AA-E5P5 sold by the company Bimax; and Maxemul® 5010 and 5011 sold by the company Uniqema.

According to the manufacturers:

Emulsogen® R109 is a random ethoxylated/propoxylated 1,4-butanediol vinyl ether having the empirical formula:

CH₂═CH—O(CH₂)₄O(C₃H₆O)₄(C₂H₄O)₁₀H;

Emulsogen® R208 is a random ethoxylated/propoxylated 1,4-butanediol vinyl ether having the empirical formula:

CH₂═CH—O(CH₂)₄O(C₃H₆O)₄(C₂H₄O)₂₀H;

Emulsogen® R307 is a random ethoxylated/propoxylated 1,4-butanediol vinyl ether having the empirical formula:

CH2=CH—O(CH2)4O(C3H6O)4(C2H4O)30H;

Emulsogen® RAL 109 is a random ethoxylated/propoxylated allylic ether having the empirical formula:

CH₂═CHCH₂—O(C₃H₆O)₄(C₂H₄O)₁₀H;

Emulsogen® RAL 208 is a random ethoxylated/propoxylated allylic ether having the empirical formula:

CH₂═CHCH₂—O(C₃H₆O)₄(C₂H₄O)₂₀H;

Emulsogen® RAL 307 is a random ethoxylated/propoxylated allylic ether having the empirical formula:

CH₂═CHCH₂—O(C₃H₆O)₄(C₂H₄O)₃₀H;

Maxemul® 5010 is a hydrophobic carboxylated C₁₂-C₁₅ alkenyl, ethoxylated with 24 ethylene oxide units,

Maxemul® 5011 is a hydrophobic carboxylated C₁₂-C₁₅ alkenyl, ethoxylated with 34 ethylene oxide units; and

BX-AA-E5P5 is a random ethoxylated/propoxylated allylic ether having the empirical formula:

CH₂═CHCH₂—O(C₃H₆O)₆(C₂H₄O)₅H.

The amount of at least one semi-hydrophobic vinyl surfactant monomer used in the preparation of the at least one cationic polymer (i) used in the composition according to the present disclosure may vary widely and also depends on the final Theological properties desired for the polymer.

When present, the at least one semi-hydrophobic vinyl surfactant monomer represents from 0.01% to 25% by weight and even from 0.1% to 10% by weight relative to the total weight of the monomer mixture.

The at least one cationic polymer used in the composition according to the present disclosure is prepared from a monomer mixture that may comprise one or more hydroxylated nonionic vinyl monomers.

These monomers are ethylenically unsaturated monomers comprising one or more hydroxyl substituents.

Nonlimiting examples of the at least one hydroxylated nonionic vinyl monomers include hydroxylated C₁-C₆ alkyl (meth)acrylates, preferably hydroxylated C₁-C₄ alkyl (meth)acrylates, such as 2-hydroxyethyl methacrylate (HEMA), 2-hydroxyethyl acrylate (2-HEA) and 3-hydroxypropyl acrylate; hydroxylated C₁-C₄ alkyl(meth)acrylamides, such as N-(2-hydroxyethyl)methacrylamide, N-(2-hydroxyethyl)acrylamide, N-(3-hydroxypropyl)acrylamide and N-(2,3-dihydroxypropyl)acrylamide. Mention may also be made of allyl alcohol, glyceryl monoallyl ether, 3-methyl-3-buten-1-ol, and vinyl alcohol precursors and equivalents thereof, such as vinyl acetate.

When present, the at least one hydroxylated nonionic vinyl monomer generally represents up to 10% by weight relative to the total weight of the monomer mixture. In one aspect of the disclosure, the at least one hydroxylated nonionic vinyl monomer represents from 0.01% to 10% by weight, from 1% to 8%, and even from 1% to 5% by weight relative to the total weight of the monomer mixture.

The at least one cationic polymer (i) used in the composition according to the present disclosure is prepared from a monomer mixture that may comprise one or more crosslinking monomers for introducing branches and controlling the molecular mass.

Polyunsaturated crosslinking agents that may be used are well known in the art. Monounsaturated compounds with a reactive group capable of crosslinking a copolymer formed before, during, or after the polymerization may also be used. Other crosslinking monomers that may be used include polyfunctional monomers comprising multiple reactive groups such as peroxide and isocyanate groups and hydrolysable silane groups. Many polyunsaturated compounds may be used to generate a partially or substantially crosslinked three-dimensional network.

Nonlimiting examples of polyunsaturated crosslinking monomers that may be used include polyunsaturated aromatic monomers, such as divinylbenzene, divinyinaphthalene and trivinylbenzene; polyunsaturated alicyclic monomers such as 1,2,4-trivinylcyclohexane; difunctional phthalic acid esters such as diallyl phthalate; polyunsaturated aliphatic monomers such as dienes, trienes and tetraenes, including isoprene, butadiene, 1,5-hexadiene, 1,5,9-decatriene, 1,9-decadiene and 1,5-heptadiene.

Nonlimiting examples of other polyunsaturated crosslinking monomers that may be used include polyalkenyl ethers such as triallylpentaerythritol, diallylpentaerythritol, diallylsucrose, octaallylsucrose and trimethylolpropane diallyl ether; polyunsaturated esters of polyalcohols or of polyacids, such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate and polyethylene glycol di(meth)acrylate, alkylenebisacrylamides, such as methylenebisacrylamide and propylenebisacrylamide; hydroxylated and carboxylated derivatives of methylenebisacrylamide, such as N,N′-bismethylol methylenebisacrylamide; polyethylene glycol di(meth)acrylates, such as ethylene glycol di(meth)acrylate, diethylene glycol di(meth)acrylate and triethylene glycol di(meth)acrylate, polyunsaturated silanes such as dimethyldivinylsilane, methyltrivinylsilane, allyldimethylvinylsilane, diallyldimethylsilane and tetravinylsilane, and polyunsaturated stannanes, such as tetraallyltin and diallyidimethyltin.

Nonlimiting examples of monounsaturated crosslinking monomers that may be used and that bear a reactive group include N-methylolacrylamides; N-alkoxy(meth)acrylamides, in which the alkoxy group is a C₁-C₁₈ group; and unsaturated hydrolysable silanes such as triethoxyvinylsilane, tris-isopropoxyvinylsilane and 3-triethoxysilylpropyl methacrylate.

Nonlimiting examples of polyfunctional crosslinking monomers that may be used and that comprise several reactive groups include hydrolysable silanes such as ethyltriethoxysilane and ethyltrimethoxysilane; epoxidized hydrolysable silanes such as 2-(3,4-epoxycyclohexyl)ethyltriethoxysilane and 3-glycidoxypropyltrimethyoxysi lane; polyisocyanates, such as 1,4-diisocyanatobutane, 1,6-diisocyanatohexane, 1,4-phenylenediisocyanate and 4,4′-oxybis(phenyl)socyanate); unsaturated epoxides, such as glycidyl methacrylate and allyl glycidyl ether; polyepoxides, such as diglycidyl ether, 1,2,5,6-diepoxyhexane and ethylene glycol diglycidyl ether.

In one aspect of the disclosure, polyunsaturated crosslinking monomers that may be used are ethoxylated polyols, such as diols, triols and bis-phenols, ethoxylated with 2 to 100 mol of ethylene oxide per mole of hydroxyl functional group and ending with a polymerizable unsaturated group such as a vinyl ether, an allyl ether, an acrylate ester or a methacrylate ester. Such crosslinking monomers may be, for example, ethoxylated bisphenol A dimethacrylate, ethoxylated bisphenol F dimethacrylate and ethoxylated trimethylolpropane trimethacrylate.

Other ethoxylated crosslinking monomers that may be used in another aspect of the present disclosure are, for example, the crosslinking agents derived from ethoxylated polyols disclosed in U.S. Pat. No. 6,140,435.

In one embodiment of the disclosure, crosslinking monomers are chosen from acrylate and methacrylate esters of polyols containing at least two acrylate or methacrylate ester groups, such as trimethylolpropane triacrylate (TMPTA), trimethylolpropane dimethacrylate, triethylene glycol dimethacrylate (TEGDMA) and ethoxylated (30) bisphenol A dimethacrylate (EOBDMA).

In one aspect of the disclosure, when present, the at least one crosslinking monomer may represent not more than 5% by weight relative to the weight of the monomer mixture. According to one embodiment, at least one the crosslinking monomer is present in a content ranging from 0.001% to 5% by weight, from 0.05% to 2% by weight, and even from 0.1% to 1% by weight relative to the total weight of the monomer mixture.

The monomer mixture may comprise one or more chain-transfer agents. Chain-transfer agents are well known in the art.

Nonlimiting mention may be made of thiol compounds, disulfide compounds such as C₁-C₁₈ mercaptans, mercaptocarboxylic acids, mercaptocarboxylic acid esters, thioesters, C₁-C₁₈ alkyl disulfides, aryl disulfides, polyfunctional thiols; phosphites and hypophosphites; haloalkyl compounds such as carbon tetrachloride and bromotrichloromethane; and unsaturated chain-transfer agents, such as α-methylstyrene.

The polyfunctional thiols are, for example, trifunctional thiols, such as trimethylolpropane tris(3-mercaptopropionate); tetrafunctional thiols, such as pentaerythritol tetrakis(thioglycolate) and pentaerythritol tetrakis(thiolactate); and hexafunctional thiols, such as pentaerythritol hexakis(thioglycolate).

Alternatively, the chain-transfer agents(s) may be catalytic chain-transfer agents that reduce the molecular weight of the addition polymers during the free-radical polymerization of the vinyl monomers. Examples include cobalt complexes, such as cobalt (II) chelates. The catalytic chain-transfer agents may also be used at low concentrations relative to the thiolated chain-transfer agents.

Nonlimiting examples of, chain-transfer agents include octyl mercaptan, n-dodecyl mercaptan, t-dodecyl mercaptan, hexadecyl mercaptan, octadecyl mercaptan (ODM), isooctyl 3-mercaptopropionate (IMP), butyl 3-mercaptopropionate, 3-mercaptopropionic acid, butyl thioglycolate, isooctyl thioglycolate and dodecyl thioglycolate.

In one aspect of the disclosure, when present, the chain-transfer agent(s) is (are) added to the monomer mixture at up to 10% by weight relative to the total weight of the monomer mixture, or even from 0.1% to 5% by weight relative to the total weight of monomers.

The monomer mixture for preparing the at least one cationic polymer (i) used in the composition according to the present disclosure may comprise one or more polymeric stabilizers for obtaining stable dispersions or emulsions. Preferably, the polymers are water-soluble. Examples include, but are not limited to, synthetic polymers, such as polyvinyl alcohols, partially hydrolysed polyvinyl acetates, polyvinylpyrrolidone, polyacrylamides, polymethacrylamides, carboxylated addition polymers, polyalkyl vinyl ethers, water-soluble natural polymers, such as gelatin, pectins, alginates and casein; modified natural polymers, such as methylcellulose, hydroxypropylcellulose, carboxymethylcellulose and allylic hydroxyethylcelluloses.

The polymeric stabilizers are used in an amount of not more than 2% by weight relative to the total weight of the emulsion, an amount of between 0.0001% and 1% by weight, and even between 0.01% and 0.5% by weight relative to the weight of the monomer mixture.

According to one embodiment, the monomer mixture comprises, relative to the total weight of the monomer mixture:

a) from 10% to 70% by weight of at least one vinyl monomer substituted with one or more amino group,

b) from 20% to 80% by weight of at least one hydrophobic nonionic vinyl monomer,

c) from 0.001% to 25% by weight of at least one associative vinyl monomer,

d) from 0 to 25% by weight of at least one semi-hydrophobic vinyl surfactant monomer,

e) from 0 to 10% by weight of at least one hydroxylated nonionic vinyl monomer,

f) from 0 to 5% by weight of at least one crosslinking monomer,

g) from 0 to 10% by weight of at least one chain-transfer agent, and

h) from 0 to 2% by weight of at least one polymeric stabilizer.

Even more preferably, the monomer mixture comprises, relative to the total weight of the monomer mixture:

a) from 20% to 60% by weight of at least one vinyl monomer substituted with one or more amino group,

b) from 20% to 70% by weight of at least one hydrophobic nonionic vinyl monomer,

c) from 0.01% to 15% by weight of at least one associative vinyl monomer,

d) from 0.1% to 10% by weight of at least one semi-hydrophobic vinyl surfactant monomer,

e) from 0.01% to 10% by weight of at least one hydroxylated nonionic vinyl monomer,

f) from 0.001% to 5% by weight of at least one crosslinking monomer,

g) from 0.001% to 10% by weight of at least one chain-transfer agent, and

h) from 0 to 2% by weight of at least one polymeric stabilizer.

According to one particular embodiment, the monomer mixture for preparing the at least one cationic polymer (i) used in the composition according to the present disclosure comprises, relative to the total weight of the monomer mixture:

a) from 20% to 50% by weight of at least one vinyl monomer substituted with one or more amino group chosen from:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate,

b) from 50% to 65% by weight of at least one hydrophobic nonionic vinyl monomer chosen from C₁-C₃₀ alkyl esters of acrylic acid and C₁-C₃₀ alkyl esters of methacrylic acid,

c) from 0.1% to 10% by weight of at least one associative vinyl monomer chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, and polyethoxylated canola (meth)acrylates and polyethoxylated cholesteryl (meth)acrylates,

d) from 0.1% to 10% by weight of at least one semi-hydrophobic vinyl surfactant monomer having one of the following formulae:

CH₂═CH—O(CH₂)ₐO(C₃H₆O)_{b}(C₂H₄O)_{c}H or

CH₂═CHCH₂O(C₃H₆O)_{d}(C₂H₄O)ₑH;

in which:

a is equal to 2, 3 or 4;

b is an integer ranging from 1 to 10;

c is an integer ranging from 5 to 50;

d is an integer ranging from 1 to 10; and

e is an integer ranging from 5 to 50;

e) up to 10% by weight of at least one hydroxylated nonionic vinyl monomer,

f) up to 5% by weight of at least one crosslinking monomer,

g) up to 10% by weight of at least one chain-transfer agent, and

h) up to 2% by weight of at least one polymeric stabilizer.

In one aspect of the disclosure, the at least one cationic polymer (i) is derived from the polymerization of the following monomer mixture:

a di(C₁-C₄ alkyl)amino(C₁-C₅ alkyl)methacrylate,

one or more C₁-C₃₀ alkyl esters of (meth)acrylic acid,

a C₁₀-C₃₀ alkyl methacrylate polyethoxylated with 20 to 30 mol of ethylene oxide,

a 30/5 polyethylene glycol/polypropylene glycol allyl ether,

a hydroxy(C₂-C₆ alkyl)methacrylate, and

an ethylene glycol dimethacrylate.

In one embodiment of the present disclosure, the at least one cationic polymer (i) used in the composition is made of the compound sold by the company Noveon under the name Carbopol Aqua CC Polymer® and which corresponds to the INCI name Polyacrylate-1 Crosspolymer.

Polyacrylate-1 Crosspolymer is the product of polymerization of a monomer mixture comprising (or constituted of):

a di(C₁-C₄ alkyl)amino(C₁-C₆ alkyl)methacrylate,

one or more C₁-C₃₀ alkyl esters of (meth)acrylic acid,

a polyethoxylated C₁₀-C₃₀ alkyl methacrylate (20-25 mol of ethylene oxide units),

a 30/5 polyethylene glycol/polypropylene glycol allyl ether,

a hydroxy(C₂-C₆ alkyl)methacrylate, and

an ethylene glycol dimethacrylate.

The at least one cationic polymer (i) used in the compositions according to the present disclosure generally represents from 0.01% to 10% by weight, from 0.05% to 5% by weight, and even from 0.1% to 1% by weight relative to the total weight of the composition.

The at least one cationic polymer (i) used in the composition according to the present disclosure may be prepared via conventional polymerization techniques, such as emulsion polymerization, as is well known in the field of polymers. For example, the polymerization may be performed via a simple batch process, or via a controlled addition process, or alternatively the reaction may be initiated in a small reactor and the mass of monomers may then be added in a controlled manner to the reactor (seeding process). Generally, the polymerization is performed at a reaction temperature of between 20 and 80° C., although higher or lower temperatures may be used. To facilitate the emulsification of the monomer mixture, the emulsion polymerization is performed in the presence of a surfactant that is present in an amount ranging from 1% to 10% by weight, from 3% to 8% by weight, and even from 5% to 7% by weight relative to the total weight of the emulsion. The emulsion polymerization reaction medium also comprises one or more radical initiator, which may be present in an amount ranging from 0.01% to 3% by weight relative to the total weight of the monomer mixture. The polymerization may be performed in an aqueous or aqueous-alcoholic medium at a neutral or weakly alkaline pH.

In a polymerization in one aspect of the disclosure, the monomer mixture is added with stirring to a solution of emulsifying surfactants, such as a nonionic surfactant, including linear or branched alcohol ethoxylate, or a mixture of nonionic and anionic surfactants, such as fatty alkyl sulfates or alkyl sulfonates of fatty alcohols, in a suitable amount of water, in a suitable reactor, to prepare the monomer emulsion. The emulsion is deoxygenated via any known method, and the polymerization reaction is then initiated by adding a polymerization catalyst (initiator) such as sodium persulfate, or any other suitable addition polymerization catalyst, as is well known in the field of polymers. The reaction is stirred until the polymerization is complete, generally for a time ranging from 4 hours to 16 hours. The monomer emulsion may be heated to a temperature of between 20 and 80° C. before adding the initiator, if so desired. The amount of unreacted monomers may be removed by adding an additional amount of catalyst. The polymer emulsion obtained may be discharged from the reactor and packaged for storage or used. Optionally, the pH or other physical or chemical characteristics of the emulsion may be adjusted before discharging the emulsion from the reactor. Generally, the emulsion produced has a total solids content ranging between 10% and 40% by weight. Generally, the total amount of polymers in the emulsion obtained ranges between 15% and 35% by weight and, in one aspect, not more than 25% by weight.

Surfactants that are suitable for facilitating the emulsion polymerization may be nonionic, anionic, amphoteric or cationic surfactants, or mixtures thereof. In one aspect of the disclosure, nonionic or anionic surfactants, or mixtures thereof, are used.

Any type of nonionic, anionic, amphoteric or cationic surfactant conventionally used in emulsion polymerizations may be used.

The polymerization may be performed in the presence of at least one free-radical initiator. As nonlimiting examples, the at least one initiator may be chosen from insoluble inorganic persulfate compounds, such as ammonium persulfate, potassium persulfate or sodium persulfate; peroxides such as hydrogen peroxide, benzoyl peroxide, acetyl peroxide and lauryl peroxide; organic hydroperoxides, such as cumene hydroperoxide and t-butyl hydroperoxide; organic peracids, such as peracetic acid; and oil-soluble free-radical generators, such as 2,2′-azobisisobutyronitrile. The peroxides and peracids may be optionally activated with reducing agents, such as sodium bisulfite or ascorbic acid, transition metals or hydrazine. Free-radical initiators that are particularly suitable are water-soluble azo polymerization initiators such as 2,2′-azobis(tert-alkyl) compounds bearing a water-solubilizing substituent on the alkyl group. In one aspect of the disclosure, azo polymerization catalysts are the Vazo® free-radical initiators sold by the company DuPont, such as Vazo® 44 (2,2′-azobis(2-4,5-dihydroimidazolyl)propane), Vazo® 56 (2,2′-azobis(2-methylpropionamidine) dihydrochloride) and Vazo® 68 (4,4′-azobis(4-cyanovaleric acid)).

The at least one cyclodextrin includes oligosaccharides of formula (VI):

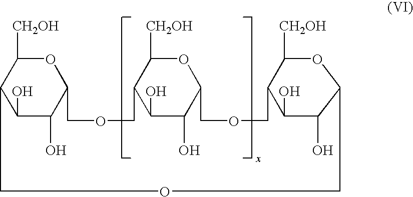

in which x may be a number equal to 4 (which corresponds to α-cyclodextrin), 5(β-cyclodextrin) or 6 (γ-cyclodextrin).

In one embodiment of the disclosure, a beta-cyclodextrin sold by the company Wacker under the name Cavamax® W7 and a gamma-cyclodextrin sold by the company Wacker under the name Cavamax® W8 may be used.

For the purposes of the present disclosure, the term “cyclodextrins” also means substituted cyclodextrin derivatives, for instance methyl cyclodextrins, such as the methyl-beta-cyclodextrin sold by the company Wacker under the name Cavasol® W7.

In one aspect of the disclosure, the at least one cyclodextrin of the disclosure is unsubstituted. In one embodiment, a beta-cyclodextrin will be used.

According to the disclosure, the at least one cyclodextrin may represent from 0.2% to 30% by weight, from 0.5% to 15% by weight, from 1% to 10% by weight and even from 1.5% to 5% by weight relative to the total weight of the final composition.

The compositions of the present disclosure also comprise one or more surfactants that may be present in an amount ranging from 0.2% to 40% by weight, from 1% to 35% and even from 1.5% to 30% relative to the total weight of the composition.

The at least one cyclodextrin and the at least one surfactant are preferably present in a concentration that is effective for making the composition nacreous and/or for forming an insoluble complex in the composition between the at least one cyclodextrin and the at least one surfactant.

The surfactant/cyclodextrin ratio may range from 0.01 to 300, preferably from 0.1 to 100 and more preferably from 0.3 to 25.

In one aspect of the disclosure, the at least one surfactant suitable for use in the present disclosure may be of any nature and is preferably soluble in water at room temperature.

(i) Anionic Surfactant(s):

As examples of anionic surfactants which can be used, alone or as mixtures, in the context of the present disclosure, nonlimiting mention is made of salts (including alkaline salts, especially sodium salts, ammonium salts, amine salts, amino alcohol salts or magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamido sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 8 to 24 carbon atoms, and the aryl radical preferably denoting a phenyl or benzyl group. Among the anionic surfactants which can also be used, nonlimiting mention is also made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants may also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 ethylene oxide groups, and mixtures thereof.

In one aspect of the disclosure, the anionic surfactant is chosen from alkyl sulfate salts and alkyl ether sulfate salts and mixtures thereof.

(ii) Nonionic Surfactant(s):

Nonionic surfactants are compounds that are well known per se (see in particular “Handbook of Surfactants” by M. R. Porter, published by Blackie & Son (Glasgow and London), 1991, pp. 116-178) and, in the context of the present disclosure, their nature is not a critical feature. Thus, as nonlimiting example, they can be chosen from polyethoxylated, polypropoxylated and polyglycerolated fatty acids, alkylphenols, α-diols or alcohols, all these compounds having a fatty chain containing, for example, 8 to 18 carbon atoms, 20 to 50 ethylene oxide or propylene oxide groups, and 2 to 30 glycerol groups. Mention is also made of copolymers of ethylene oxide and of propylene oxide, condensates of ethylene oxide and of propylene oxide with fatty alcohols; polyethoxylated fatty amides, including those having from 2 to 30 mol of ethylene oxide, polyglycerolated fatty amides containing 1 to 5, and even 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It is noted that the alkylpolyglycosides and the oxyethylenated fatty acid esters of sorbitan having in one embodiment from 2 to 10 mol of ethylene oxide constitute nonionic surfactants that are suitable in the context of the present disclosure in at least one embodiment.

In another embodiment of the present disclosure, the compositions comprise sorbitan laurate oxyethylenated with 4 mol of ethylene oxide (Tween® 21 from Uniqema).

(iii) Amphoteric or Zwitterionic Surfactant(s):

Nonlimiting examples of the amphoteric or zwitterionic surfactants include aliphatic secondary or tertiary amine derivatives in which the aliphatic radical is a linear or branched chain containing 8 to 18 carbon atoms and containing at least one water-soluble anionic group (for example carboxylate, sulfonate, sulfate, phosphate or phosphonate). Mention is also made of (C₈-C₂₀)alkylbetaines, sulfobetaines, (C₈-C₂₀)alkylamido-(C₁-C₆)alkylbetaines or (C₈-C₂₀)alkylamido(C₁-C₆)alkylsulfobetaines.

Among the amine derivatives, nonlimiting mention is made of the products sold under the name Miranol®, as described in U.S. Pat. No. 2,528,378 and U.S. Pat. No. 2,781,354 and classified in the CTFA dictionary, 3rd edition, 1982, under the names Amphocarboxyglycinates and Amphocarboxypropionates, having the respective structures:

R₂—CONHCH₂CH₂—N(R₃)(R₄)(CH₂COO—) (1)

in which: R₂ denotes an alkyl radical of an acid R₂—COOH present in hydrolysed coconut oil, a heptyl, nonyl or undecyl radical, R₃ denotes a β-hydroxyethyl group and R₄ denotes a carboxymethyl group;

and

R₅—CONHCH₂CH₂—N(B)(C) (2)

in which:

B represents —CH₂CH₂OX′, C represents —(CH₂)_{z}—Y′, with z=1 or 2,

X′ denotes the —CH₂CH₂—COOH group or a hydrogen atom,

Y′ denotes —COOH or the —CH₂—CHOH—SO₃H radical,

R₅ denotes an alkyl radical of an acid R₉—COOH present in coconut oil or in hydrolysed linseed oil, an alkyl radical, in particular a C₇, C₉, C₁₁ or C₁₃ alkyl radical, a C₁₇ alkyl radical and its iso form, an unsaturated C₁₇ radical, and

R₉ denotes alkyl radical derived from flax or coco.

By way of example, mention may be made of the cocoamphodiacetate sold under the trade name Miranol® C2M concentrated NP by the company Rhodia Chimie.

(iv) Cationic Surfactants

The cationic surfactants may be chosen from:

A) the quaternary ammonium salts of general formula (XII) below:

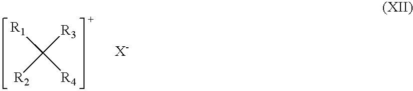

wherein X⁻ is an anion chosen from the group of halides (chloride, bromide or iodide) or (C₂-C₆)alkyl sulfates, more particularly methyl sulfate, phosphates, alkyl or alkylaryl sulfonates, anions derived from organic acid, such as acetate or lactate, and

the radicals R₁ to R₃, which may be identical or different, represent a linear or branched aliphatic radical containing from 1 to 4 carbon atoms, or an aromatic radical such as aryl or alkylaryl. The aliphatic radicals can comprise heteroatoms such as oxygen, nitrogen, sulfur or halogens, The aliphatic radicals are chosen, for example, from alkyl, alkoxy and alkylamide radicals,

R₄ denotes a linear or branched alkyl radical containing from 16 to 30 carbon atoms.

In one embodiment, the cationic surfactant is a cetyltrimethylammonium salt (for example chloride).

In another aspect of the disclosure, the cationic surfactants may be chosen from the quaternary ammonium salts of general formula (XII) above wherein:

X⁻ is an anion chosen from the group of halides (chloride, bromide or iodide) or (C₂-C₆)alkyl sulfates, more particularly methyl sulfate, phosphates, alkyl or alkylaryl sulfonates, anions derived from organic acid, such as acetate or lactate, and

the radicals R₁ and R₂, which may be identical or different, represent a linear or branched aliphatic radical containing from 1 to 4 carbon atoms, or an aromatic radical such as aryl or alkylaryl. The aliphatic radicals can comprise heteroatoms such as oxygen, nitrogen, sulfur or halogens. The aliphatic radicals are chosen, for example, from alkyl, alkoxy, alkylamide and hydroxyalkyl radicals containing from about 1 to 4 carbon atoms;

R₃ and R₄, which may be identical or different, denote a linear or branched alkyl radical containing from 12 to 30 carbon atoms, with the radical comprising at least one ester or amide function.

R₃ and R₄ are chosen in particular from (C₁₂-C₂₂)alkylamido(C₂-C₆)alkyl and (C₁₂-C₂₂)alkylacetate radicals.

In one embodiment of the disclosure, the cationic surfactant is a stearamidopropyldimethyl(myristyl acetate)ammonium salt (for example chloride).

B)—the quaternary ammonium salts of imidazolinium, such as, for example, that of formula (XIII) below:

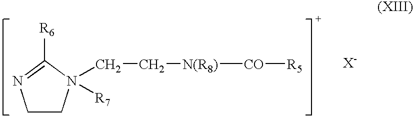

in which R₅ represents an alkenyl or alkyl radical containing from 8 to 30 carbon atoms, for example fatty acid derivatives of tallow, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, and X⁻ is an anion chosen from the group of halides, phosphates, acetates, lactates, alkyl sulfates, alkyl sulfonates or alkylaryl sulfonates. In one aspect of the disclosure, R₅ and R₆ denote a mixture of alkenyl or alkyl radicals containing from 12 to 21 carbon atoms, such as, for example, fatty acid derivatives of tallow, R₇ denotes methyl and R₈ denotes hydrogen. Such a product is, for example, Quaternium-27 (CTFA 1997) or Quaternium-83 (CTFA 1997), which are sold under the names Rewoquat® W75, W90, W75PG and W75HPG by the company Witco,

C)—the diquaternary ammonium salts of formula (IX):

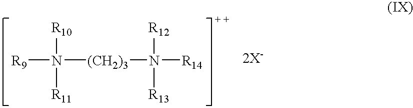

in which R₉ denotes an aliphatic radical containing from about 16 to 30 carbon atoms, R₁₀, R₁₁, R₁₂, R₁₃ and R₁₄, which may be identical or different, are chosen from hydrogen and alkyl radicals containing from 1 to 4 carbon atoms, and X⁻ is an anion chosen from the group of halides, acetates, phosphates, nitrates and methyl sulfates. Such diquaternary ammonium salts in particular may comprise propanetallowediammonium dichloride;

D)—the quaternary ammonium salts containing at least one ester function, of formula (X) below:

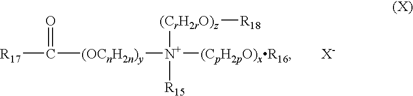

in which:

R₁₅ is chosen from C₁-C₈ alkyl radicals and C₁-C₆ hydroxyalkyl or dihydroxyalkyl radicals,

R₁₆ is chosen from:

a radical R₁₉

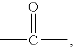

linear or branched, saturated or unsaturated C₁-C₂₂ hydrocarbon radicals R₂₀, and a hydrogen atom;

R₁₈ is chosen from:
a radical R₂₁

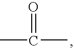

linear or branched, saturated or unsaturated C₁-C₆ hydrocarbon radicals R₂₂, and a hydrogen atom;

R₁₇, R₁₉ and R₂₁, which may be identical or different, are chosen from linear or branched, saturated or unsaturated C₇-C₂₁ hydrocarbon radicals;

R₂₀ is chosen from linear or branched, saturated or unsaturated C₁-C₂₂ hydrocarbon radicals;

R₂₂ is chosen from linear or branched, saturated or unsaturated C₁-C₆ hydrocarbon radicals;

n, p and r, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₁₆ denotes R₂₀ and that when z is 0, then R₁₈ denotes R₂₂.

In one embodiment of the disclosure, the ammonium salts are of formula (X) in which:

R₁₅ denotes a methyl or ethyl radical,

x and y are equal to 1;

z is equal to 0 or 1;

n, p and r are equal to 2;

R₁₆ is chosen from:

a radical R₁₉

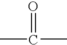

methyl, ethyl or C₁₄-C₂₂ hydrocarbon radicals,

a hydrogen atom;

R₁₇, R₁₉ and R₂₁, which may be identical or different, are chosen from linear or branched, saturated or unsaturated C₇-C₂₁ hydrocarbon radicals; and

R₁₈ is chosen from:

a radical R₂₁

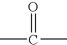

a hydrogen atom.

Such compounds are sold, for example, under the names Dehyquart® by the company Cognis, Stepanquat® by the company Stepan, Noxamium® by the company Ceca, and Rewoquat® WE 18 by the company Degussa-Witco.

In one embodiment of the disclosure, the quaternary ammonium salts are cetyltrimethylammonium chloride and palmitamidopropyltrimethylammonium chloride, sold under the name Varisoft® PA TC by the company Degussa.

In one aspect of the present disclosure, the compositions comprise at least one anionic surfactant. The at least one anionic surfactant is generally present in concentrations ranging from 3% to 30% by weight and even from 5% to 20% by weight relative to the total weight of the composition.

In at least one embodiment of the disclosure, the anionic surfactant used is sodium, triethanolamine or ammonium (C₁₂-C₁₄)alkyl sulfates, sodium, triethanolamine or ammonium (C₁₂-C₁₄)alkyl ether sulfates oxyethylenated with 2.2 mol of ethylene oxide, sodium cocoyl isethionate and sodium α-(C₁₄-C₁₆)olefin sulfonate, and mixtures thereof with:

either an amphoteric surfactant such as the amine derivatives known as disodium cocoamphodiacetate or sodium cocoamphopropionate sold especially by the company Rhodia Chimie under the trade name Miranol® C2M Conc NP as an aqueous solution containing 38% active material, or under the name Miranol® C32;

or an amphoteric surfactant, such as alkylbetaines in particular the cocobetaine sold under the name Dehyton® AB 30 as an aqueous solution containing 32% AM by the company Cognis, or such as (C₈-C₂₀)alkylamido(C₁-C₆)alkylbetaines, in particular Tegobetaine® F 50 sold by the company Degussa.

The amphoteric or nonionic surfactants may be present in concentrations ranging from 0.5% to 20% by weight and even from 1% to 15% by weight relative to the total weight of the composition.

According to one variant of the present disclosure, the cosmetic compositions may also comprise one or more keratin material conditioning agent.

When the composition contains at least one conditioning agent, these agents are generally chosen from synthetic oils such as poly-α-olefins, fluoro oils, fluoro waxes, fluoro gums, carboxylic acid esters, cationic polymers other than those of the present disclosure, silicones, mineral, plant or animal oils, ceramides and pseudoceramides, and mixtures thereof.

The polyolefins may be poly-α-olefins and in particular, but not limited to:

of hydrogenated or non-hydrogenated polybutene type, including hydrogenated or non-hydrogenated polyisobutene.

In one aspect of the disclosure, isobutylene oligomers with a molecular weight of less than 1000 and mixtures thereof with polyisobutylenes with a molecular weight of greater than 1000 and even ranging from 1000 to 15 000.may be used.

Nonlimiting examples of poly-α-olefins that may be used in the context of the present disclosure are the polyisobutenes sold under the name Permethyl® 99 A, 101 A, 102 A′, 104 A (n=16) and 106 A (n=38) by the company Presperse Inc., or alternatively the products sold under the name Arlamol HDO (n=3) by the company ICI (n denoting the degree of polymerization),

of hydrogenated or non-hydrogenated polydecene type.

Such products are sold, for example, under the names Ethylflo® by the company Ethyl Corp. and Arlamo®I PAO by the company ICI.

The mineral oils that may be used in the compositions of the present disclosure include those chosen from the group formed by:

hydrocarbons such as hexadecane and liquid paraffin.

The at least one cationic polymer that may be used in accordance with the present disclosure may be chosen from all those that are already known as improving the cosmetic properties of hair treated with detergent compositions, i.e. including, but not limited to, those described in patent application EP-A-0 337 354 and in French patent applications FR-A-2 270 846, 2 383 660, 2 598 611, 2 470 596 and 2 519 863.

For the purposes of the present disclosure, the term “cationic polymer” denotes any polymer containing cationic groups and/or groups that may be ionized into cationic groups.

Among the cationic polymers that may be used in the context of the present disclosure, one aspect of the disclosure uses quaternary cellulose ether derivatives such as the products sold under the name JR 400 by the company Amerchol, cyclopolymers, including diallyldimethylammonium salt homopolymers and copolymers of a diallyldimethylammonium salt and of acrylamide, including the chlorides, sold under the names Merquat® 100, Merquat® 550 and Merquate® S by the company Nalco, cationic polysaccharides and in one embodiment guar gums modified with 2,3-epoxypropyl-trimethylammonium chloride, sold, for example, under the name Jaguar® C13S by the company Meyhall, optionally crosslinked homopolymers and copolymers of a (meth)acryloyloxyethyltrimethylammonium salt, sold by the company Ciba as a 50% solution in mineral oil under the trade names Salcare® SC92 (crosslinked copolymer of methacryloyloxyethyltrimethylammonium chloride and of acrylamide) and Salcare® SC95 (crosslinked methacryloyloxyethyltrimethylammonium chloride homopolymer).

It is also possible to use polymers that are constituted of repeating units corresponding to the formula:

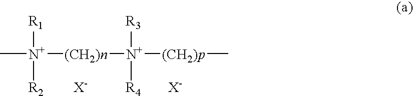

in which R₁, R₂, R₃ and R₄, which may be identical or different, denote an alkyl or hydroxyalkyl radical containing from 1 to 4 carbon atoms, n and p are integers ranging from 2 to 20, and X⁻ is an anion derived from a mineral or organic acid.

The silicones that may be used in accordance with the present disclosure include, but are not limited to, polyorganosiloxanes that are insoluble in the composition and may be in the form of oils, waxes, resins or gums.

The water-insoluble silicones are insoluble in water at a concentration of greater than or equal to 0.1% by weight in water at 25° C., i.e. they do not form a transparent isotropic solution.

The viscosity of the silicones is measured, for example, at 2500 according to ASTM standard 445 Appendix C.

The organopolysiloxanes are defined in greater detail in Walter Noll's “Chemistry and Technology of Silicones” (1968) Academic Press. They may be volatile or non-volatile.

When they are volatile, the silicones may be chosen from, but are not limited to, those having a boiling point ranging from 60° C. to 260° C., and include those chosen from:

(i) cyclic silicones containing from 3 to 7 and even 4 to 5 silicon atoms. For example, octamethylcyclotetrasiloxane sold in particular under the name Volatile Silicone 7207 by Union Carbide or Silbione® 70045 V 2 by Rhodia Chimie, decamethylcyclopentasiloxane sold under the name Volatile Silicone 7158 by Union Carbide, and Silbione® 70045 V 5 by Rhodia Chimie, and mixtures thereof.

Mention is also made of cyclocopolymers of the dimethylsiloxanes/methylalkylsiloxane type, such as Volatile Silicone FZ 3109 sold by the company Union Carbide, with the chemical structure:

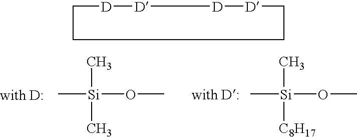

Mention is also made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

In one aspect of the present disclosure, non-volatile silicones are used, including polyalkylsiloxanes, polyarylsiloxanes, polyalkylarylsiloxanes, silicone gums and resins, polyorganosiloxanes modified with organofunctional groups, and also mixtures thereof.

These silicones are more particularly chosen from polyalkylsiloxanes, among which mention is made of polydimethylsiloxanes containing trimethylsilyl end groups having a viscosity of from 5×10⁻⁶ to 2.5 m²/s at 25° C. and preferably 1×10⁻⁵ to 1 m²/s.

Among these polyalkylsiloxanes, mention is made, in a non-limiting manner, of the following commercial products:

the oils of the Mirasil® series sold by the company Rhodia Chimie, for instance the Mirasil® DM 500 000 oil;

the oils of the 200 series from the company Dow Corning, such as DC200 with a viscosity of 60 000 cSt;

the Viscasi®I oils from General Electric and certain oils of the SF series (SF 96, SF 18) from General Electric.

Mention is also made of polydimethylsiloxanes containing dimethylsilanol end groups (Dimethiconol according to the CTFA name) such as the oils of the 48 series from the company Rhodia Chimie.

In this category of polyalkylsiloxanes, mention is also made of the products sold under the names Abil Wax® 9800 and 9801 by the company Degussa, which are poly(C₁-C₂₀)alkylsiloxanes.

In one embodiment, the polyalkylarylsiloxanes are chosen from polydimethylmethylphenylsiloxanes and linear and/or branched polydimethyldiphenylsiloxanes with a viscosity of from 1×10⁻⁵ to 5×10⁻² m²/s at 25° C.

Among these polyalkylarylsiloxanes, examples include the products sold under the following names:

the Mirasil® DPDM oils from Rhodia Chimie;

the oils of the Rhodorsil® 70 633 and 763 series from Rhodia Chimie;

the oil Dow Corning 556 Cosmetic Grade Fluid from Dow Corning;

the silicones of the PK series from Bayer, for instance the product PK20;

the silicones of the PN and PH series from Bayer, for instance the products PN1000 and PH1000;

certain oils of the SF series from General Electric, such as SF 1023, SF 1154, SF 1250 and SF 1265.

Silicone gums that may be used in accordance with the disclosure include, but are not limited to, polydiorganosiloxanes having high number-average molecular masses of between 200 000 and 1 000 000, used alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Nonlimiting mention is made of the following products:

polydimethylsiloxane,

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

In one aspect of the disclosure, products that may be used in accordance with the disclosure are mixtures such as:

mixtures formed from a polydimethylsiloxane gum hydroxylated at the chain end (known as dimethiconol according to the nomenclature of the CTFA dictionary), and from a cyclic polydimethylsiloxane (known as cyclomethicone according to the nomenclature of the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that may be used as additive are crosslinked siloxane systems containing the following units:

R₂SiO_{2/2}, R₃SiO_{1/2}, RSiO_{3/2} and SiO_{4/2} in which R represents a hydrocarbon group containing 1 to 16 carbon atoms or a phenyl group. In one aspect of the disclosure, ones in which R denotes a C₁-C₄ lower alkyl radical, including methyl, or a phenyl radical, are used.

Among these resins, nonlimiting mention may be made of the product sold under the name Dow Corning 593 or those sold under the names Silicone Fluid SS 4230 and SS 4267 by the company General Electric, which are silicones of dimethyl/trimethyl siloxane structure.

Mention is also made of the trimethyl siloxysilicate type resins sold in particular under the names X22-4914, X21-5034 and X21-5037 by the company Shin-Etsu.

The organomodified silicones that may be used in accordance with the present disclosure are silicones as defined above and containing in their structure one or more organofunctional groups attached via a hydrocarbon-based radical.

Among the organomodified silicones that may be used are polyorganosiloxanes containing:

polyethyleneoxy and/or polypropyleneoxy groups optionally containing C₆-C₂₄ alkyl groups, such as the products known as dimethicone copolyol sold by the company Dow Corning under the name DC 1248 or the oils Silwet® L 722, L 7500, L 77 and L 711 from the company Union Carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names GP 72 A and GP 71 from Genesee;

alkoxylated groups such as the product sold under the name Silicone Copolymer F-755 by SWS Silicones and Abil Wax® 2428, 2434 and 2440 by the company Degussa;

hydroxylated groups such as the polyorganosiloxanes containing a hydroxyalkyl function, described in French patent application FR-A-85 16334;

acyloxyalkyl groups such as, for example, the polyorganosiloxanes described in U.S. Pat. No. 4,957,732;

anionic groups of the carboxylic type, such as in the products described in patent EP 186 507 from the company Chisso Corporation, or of the alkylcarboxylic type, such as those present in the product X-22-3701 E from the company Shin-Etsu; 2-hydroxyalkyl sulfonate; or 2-hydroxyalkyl thiosulfate such as the products sold by the company Degussa under the names Abil® S201 and Abil® S255;

hydroxyacylamino groups, such as the polyorganosiloxanes described in patent application EP 342 834, for example the product Q2-8413 from the company Dow Corning.

According to the disclosure, it is also possible to use silicones comprising a polysiloxane portion and a portion consisting of a non-silicone organic chain, one of the two portions constituting the main chain of the polymer, the other being grafted onto the main chain. These polymers are described, for example, in patent applications EP-A-412 704, EP-A-412 707, EP-A-640 105, WO 95/00578, EP-A-582 152 and WO 93/23009 and U.S. Pat. Nos. 4,693,935, 4,728,571 and 4,972,037. In one aspect of the disclosure, these polymers are anionic or nonionic.

Such polymers are, for example, but not limited to, copolymers that can be obtained by free-radical polymerization starting with a monomer mixture consisting of:

a) 50 to 90% by weight of tert-butyl acrylate;

b) 0 to 40% by weight of acrylic acid; and

c) 5 to 40% by weight of silicone macromer of formula:

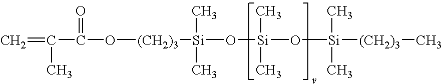

with v being a number ranging from 5 to 700; the weight percentages being calculated relative to the total weight of the monomers.

Other nonlimiting examples of grafted silicone polymers are polydimethylsiloxanes (PDMS) onto which are grafted, via a connecting chain unit of thiopropylene type, mixed polymer units of the poly(meth)acrylic acid type and of the polyalkyl (meth)acrylate type, and polydimethylsiloxanes (PDMS) onto which are grafted, via a connecting chain unit of thiopropylene type, polymer units of the polyisobutyl (meth)acrylate type.

According to the disclosure, all the silicones may likewise be used in the form of emulsions.

In one aspect of the disclosure, the polyorganosiloxanes are chosen from:

non-volatile silicones chosen from the family of polyalkylsiloxanes containing trimethylsilyl end groups, such as oils with a viscosity ranging from 0.2 to 2.5 m²/s at 25° C., such as the oils of the DC200 series from Dow Corning, including the product of viscosity 60 000 cSt, of the Mirasil® DM series, and particularly the oil Mirasil® DM 500 000, sold by the company Rhodia Chimie or the AK 300 000 silicone oil from the company Wacker, and polyalkylsiloxanes containing dimethylsilanol end groups, such as dimethiconols or polyalkylarylsiloxanes, such as the oil Mirasil® DPDM sold by the company Rhodia Chimie; and

polysiloxanes containing amine groups such as amodimethicones or trimethylsilylamodimethicones.

According to the present disclosure, the compounds of ceramide type are in particular natural or synthetic ceramides and/or glycoceramides and/or pseudoceramides and/or neoceramides.

Compounds of ceramide type are described, for example, in Patent Applications DE 4 424 530, DE 4 424 533, DE 4 402 929, DE 4 420 736, WO 95/23807, WO 94/07844, EP-A-0 646 572, WO 95/16665, FR-2 673 179, EP-A-0 227 994, WO 94/07844, WO 94/24097 and WO 94/10131, the teachings of which are included herein by way of reference.

In one aspect of the disclosure, compounds of ceramide type that are used are, for example:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalm itoyl]aminooctadecane-1,3-diol,
2-N-stearoylaminooctadecane-1,3,4-triol, including N-stearoylphytosphingosine,
2-N-palm itoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine, and

or mixtures of these compounds.

In one aspect of the disclosure, liquid fatty esters may be used as surfactants. The liquid fatty esters may be They may be, for example, monomeric, and may also be nonionic and/or non-silicone.

According to the disclosure, the term “liquid fatty esters” means an ester that is liquid at room temperature (25° C.) and at atmospheric pressure (1 atm) and that is derived from a carboxylic acid and an alcohol, with at least one of these two constituents containing at least 7 carbon atoms.

In one aspect of the disclosure, the liquid fatty esters are chosen from:

1) esters of a C₃-C₃₀ carboxylic acid and of a C₁-C₃₀ alcohol, at least one from among the acid and the alcohol being branched or unsaturated (containing at least one carbon-carbon double bond), and

2) esters of a C₇-C₃₀ aromatic acid whose carboxylic function is directly linked to the aromatic ring, and of a C₁-C₃₀ alcohol.

In another aspect of the disclosure, the esters are water-insoluble liquid esters.

The water-insoluble liquid carboxylic acid esters are insoluble in water at a concentration of greater than or equal to 0.1% by weight in water at 25° C., i.e., they do not form, under these conditions, a transparent macroscopically isotropic solution.

The total carbon number of the esters of the disclosure is generally greater than or equal to 10 and may be less than 50 and even less than 25.

In one aspect of the disclosure, the liquid esters of a C₃-C₃₀ carboxylic acid and of a C₁-C₃₀ alcohol, at least one from among the acid and the alcohol being branched or unsaturated, are chosen from esters of a C₆-C₂₄ carboxylic acid and of a C₃-C₂₀ alcohol

As nonlimiting examples, the esters according to the disclosure may be chosen from:

esters of a linear carboxylic acid containing from 12 to 26 carbon atoms and of a branched alcohol containing from 3 to 12 carbon atoms,

esters of a linear carboxylic acid containing from 2 to 12 carbon atoms and of a branched alcohol containing from 8 to 26 carbon atoms, and

esters of a branched carboxylic acid containing from 8 to 26 and preferably from 8 to 12 carbon atoms, and of a branched alcohol containing from 8 to 26 and preferably from 8 to 12 carbon atoms.

In one embodiment, esters are chosen from octyldodecyl behenate; isocetyl behenate; isocetyl lactate; isostearyl lactate; linoleyl lactate; oleyl lactate; isostearyl octanoate; isocetyl octanoate; decyl oleate; isocetyl isostearate; isocetyl laurate; isocetyl stearate; isodecyl octanoate; isodecyl oleate; isononyl isononanoate; isostearyl palmitate; myristyl isostearate; octyl isononanoate; 2-ethylhexyl isononanoate; octyl isostearate; octyidodecyl erucate; isopropyl palmitate, 2-ethylhexyl palmitate, 2-octyldecyl palmitate, branched alkyl myristates such as isopropyl myristate, t-butyl myristate or 2-octyidodecyl myristate, hexyl isostearate, butyl isostearate, isobutyl stearate; and 2-hexyldecyl laurate.

Esters of C₄-C₂₂ dicarboxylic or tricarboxylic acids and of C₁-C₂₂ alcohols and esters of monocarboxylic, dicarboxylic or tricarboxylic acids and of C₂-C₂₆ dihydroxy, trihydroxy, tetrahydroxy or pentahydroxy alcohols may also be used.

Mention is also made of: diisopropyl sebacate; diisopropyl adipate; diisostearyl adipate; octyldodecyl stearoyl stearate; pentaerythrityl tetraisononanoate; pentaerythrityl tetraisostearate; triisopropyl citrate; triisostearyl citrate; and trioctyidodecyl citrate.

Liquid esters of a branched carboxylic acid containing from 4 to 6 carbon atoms and of an alcohol containing from 8 to 26 carbon atoms may also be used.

In one aspect of the disclosure, these branched liquid esters have the following formula.

R₁COOR₂ (VI)

in which:

R₁ denotes an optionally mono- or polyhydroxylated, branched hydrocarbon-based radical containing from 3 to 5 carbon atoms, and

R₂ denotes an optionally mono- or polyhydroxylated, linear or branched hydrocarbon-based radical containing from 12 to 26 carbon atoms, or even containing from 16 to 22 carbon atoms.

In one embodiment, R₁ denotes a branched alkyl radical containing from 3 to 5 carbon atoms, and even a tert-butyl radical.

In one embodiment, R₂ denotes a saturated or unsaturated alkyl radical containing 12 to 26 carbon atoms, including branched, and even chosen from tridecyl, isocetyl, isostearyl, octyidodecyl and isoarachidyl radicals.

In another embodiment, the branched liquid esters are isostearyl neopentanoate (formula (VI) in which R₁=tert-butyl and R₂=isostearyl), tridecyl neopentanoate, isocetyl neopentanoate and isoarachidyl neopentanoate.

In another aspect of the disclosure, the acid and the alcohol of the ester are saturated, and the alcohol may be a monoalcohol (only one hydroxyl function).

Among the esters mentioned above, nonlimiting examples include isopropyl palmitate, 2-ethylhexyl palmitate, 2-octyldecyl palmitate, branched alkyl myristates such as isopropyl myristate, t-butyl myristate or 2-octyldodecyl myristate, hexyl isostearate, butyl isostearate, isobutyl stearate; 2-hexyldecyl laurate, isostearyl neopentanoate, tridecyl neopentanoate and isononyl isononanoate.

The esters of a C₇-C₃₀ aromatic acid and of a C₁-C₃₀ alcohol include esters of a C₇-C₁₇ aromatic acid and of a C₁-C₂₀ alcohol, for example, the C₁₂-C₁₅ alkyl benzoates, isostearyl benzoate, octyldodecyl benzoate, behenyl benzoate and 2-ethylhexyl benzoate.

In one embodiment, the esters are monoesters of a carboxylic acid and of a monoalcohol.

According to the disclosure, the conditioning agents may represent from 0.001% to 10% by weight, from 0.005% to 5% by weight and even from 0.01% to 3% by weight relative to the total weight of the final composition.

The physiologically acceptable medium may be constituted by water or a mixture of water and of cosmetically or dermatologically acceptable solvents such as monoalcohols, polyols or polyol ethers, which may be used alone or as a mixture. In one aspect, the water represents from 30% to 98% by weight and even from 50% to 98% by weight relative to the total weight of the composition.

Particular mention is made of monoalcohols such as ethanol and isopropanol, polyols such as diethylene glycol and glycerol, and polyol ethers such as diethylene glycol ethers.

The composition of the disclosure may also contain at least one additive chosen from sequestrants, softeners, foam modifiers, dyes, other nacreous agents, moisturizers, antidandruff or anti-seborrhoeic agents, other suspension agents, fatty acids, thickeners, fragrances, preserving agents, sunscreens, proteins, vitamins and provitamins, anionic, nonionic or amphoteric polymers, and any other additive conventionally used in cosmetics.

These additives are present in the composition according to the disclosure in proportions that may range from 0 to 40% by weight relative to the total weight of the composition. The precise amount of each additive depends on its nature and is readily determined by a person skilled in the art.

Needless to say, a person skilled in the art will take care to select the optional compound(s) to be added to the composition according to the disclosure such that the advantageous properties intrinsically associated with the composition in accordance with the disclosure are not, or are not substantially, adversely affected by the envisaged addition.

The compositions in accordance with the disclosure may be used for treating keratin materials such as the hair, the skin, the eyelashes, the eyebrows, the nails, the lips or the scalp, and more particularly the hair.

The compositions may also be used for washing and cleansing keratin materials such as the hair and the skin.

The compositions according to the disclosure are generally used as products especially for washing, caring for, conditioning, holding the style of or shaping keratin materials such as the hair.

In one aspect of the disclosure, the compositions may be in the form of shampoos, rinse-out or leave-in hair conditioners, compositions for permanent-waving, relaxing, dyeing or bleaching the hair, or alternatively in the form of compositions to be applied before or after dyeing, bleaching, permanent-waving or relaxing the hair or else between the two steps of a permanent-waving or hair-relaxing operation. In one embodiment, the compositions are washing and foaming compositions for the hair and/or the skin.

The compositions according to the disclosure can be foaming detergent compositions such as shampoos, shower gels and bubble baths, or makeup-removing products.

The minimum quantity of surfactant is that which is sufficient to give the final composition satisfactory foaming power and/or detergent power.

Thus, according to the disclosure, the detergent surfactant can represent from 3% to 30% by weight, from 6% to 25% by weight and even from 8% to 20% by weight relative to the total weight of the final composition.

The foaming power of the compositions according to the disclosure, characterized by a foam height, is generally greater than 75 mm and preferably greater than 100 mm, measured according to the modified Ross-Miles method (NF T 73-404/IS696).

The method can be modified in the following manner:

The measurement is performed at a temperature of 22° C. with osmosed water. The concentration of the solution is 2 g/l. The height of the drop is 1 m. The amount of composition that is dropped is 200 ml. These 200 ml of composition fall into a measuring cylinder 50 mm in diameter and containing 50 ml of the test composition. The measurement is taken 5 minutes after stopping the flow of the composition.

When the composition is in the form of a conditioner, which may be a rinse-out conditioner, it advantageously may contain one cationic surfactant, its concentration may range from 0.1% to 10% by weight and even from 0.5% to 5% by weight relative to the total weight of the composition.

The compositions according to the disclosure may also be in the form of aqueous or aqueous-alcoholic lotions for skincare and/or haircare.

The cosmetic compositions according to the disclosure may be in the form of a gel, a milk, a cream, an emulsion, a thickened lotion or a mousse and may be used for the skin, the nails, the eyelashes, the lips and, more particularly, the hair.

The compositions may be conditioned in various forms, especially in vaporizers, pump-dispenser bottles or in aerosol containers to allow an application of the composition in vaporized form or in the form of a mousse. Such conditioning forms are indicated, for example, when it is desired to obtain a spray, a lacquer or a mousse for treating the hair.

A subject of the disclosure is also a cosmetic process for treating keratin materials such as the hair, which consists in applying to the hair a composition as defined above and then in optionally rinsing with water, after an optional leave-in time.

As disclosed herein, the percentages stated are by weight,

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

As disclosed herein, the percentages stated are by weight.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The disclosure will now be illustrated more fully with the aid of the examples that follow, which should not be considered as limiting the disclosure to the embodiments described. In the following, AM means active material.

### EXAMPLES 1 TO 3

Shampoos having the composition below were prepared:

| | Ex. 1 | | Ex. 2 | | Ex. 3 | |
|---|---|---|---|---|---|---|
| Sodium lauryl ether sulfate (2.2 EO) | 15.5 | g AM | 15.5 | g AM | 14 | g AM |
| as an aqueous solution (Texapon AOS 225 | | | | | | |
| UP from Cognis) | | | | | | |
| Cocoyl amidopropyl betaine as an aqueous | 2.4 | g AM | 2.4 | g AM | 2.4 | g AM |
| solution (Tegobetaine F 50 from | | | | | | |
| Goldschmidt) | | | | | | |
| Copolymer of acrylic or methacrylic | 0.6 | g AM | 0.6 | g AM | 0.6 | g AM |
| acid esters, of di(C1-4 alkyl)amino(C1-6 alkyl) | | | | | | |
| methacrylate, of PEG/PPG-30/5 allyl ether, of | | | | | | |
| C10-30 PEG 20-25 alkyl ether methacrylate | | | | | | |
| and of C2-6 hydroxyalkyl methacrylate | | | | | | |
| crosslinked with ethylene glycol | | | | | | |
| dimethacrylate, as an emulsion at 20% by | | | | | | |
| weight in water (Carbopol Aqua CC Polymer | | | | | | |
| from Noveon) | | | | | | |
| Hydroxypropyl guar | 0.1 | g | 0.2 | g | | |
| trimethylammonium chloride (Jaguar C13S | | | | | | |
| from Rhodia) | | | | | | |
| Beta-cyclodextrin | 1.72 | g AM | 1.72 | g AM | 1.72 | g AM |
| (cyclomaltoheptaose) (Cavamax W7 | | | | | | |
| Pharma) | | | | | | |
| Polydimethylsiloxane | 2.7 | g | | | | |
| (DC 200 Fluid 300 000 from Dow | | | | | | |
| Corning) | | | | | | |
| Polydimethylsiloxane | | | 2.7 | g | | |
| (DC 200 Fluid 60 000 from Dow | | | | | | |
| Corning) | | | | | | |
| Polydimethylsiloxane | | | | | 1.5 | g |
| (DC 200 Fluid 500 000 from Dow | | | | | | |
| Corning) | | | | | | |
| Quaternized hydroxyethylcellulose | | | | | 0.4 | g |
| (Polymer JR 400 from Amerchol) | | | | | | |
| Oxyethylenated (20 EO) | 0.5 | g | | | | |
| oxypropylenated (5 PO) cetyl alcohol | | | | | | |
| (Procetyl AWS from Croda) | | | | | | |
| Sorbitan monolaurate oxyethylenated | | | 6 | g | 6 | g |
| with 4 mol of ethylene oxide (Tween 21 from | | | | | | |
| Uniqema) | | | | | | |
| Oxyethylenated jojoba wax (120 EO) | 0.05 | g | | | | |
| (Florasolvs Jojoba PEG 120 from | | | | | | |
| Floratech) | | | | | | |
| Isostearyl neopentanoate | 0.25 | g | | | | |
| (Ceraphyl 375 from ISP) | | | | | | |
| Sodium chloride | 2.2 | g | 1 | g | 1 | g |
| Lactic acid | 0.29 | g | 0.34 | g | 0.28 | g |
| Disodium salt of fuchsin acid D (Cl: | 0.00012 | g | | | | |
| 17200) | | | | | | |
| Powdered salicylic acid (preserving | 0.2 | g | 0.2 | g | 0.2 | g |
| agent) | | | | | | |
| Ethyl p-hydroxybenzoate (preserving | 0.15 | g | 0.15 | g | 0.15 | g |
| agent) | | | | | | |
| Sodium benzoate (preserving agent) | 0.5 | g | 0.5 | g | 0.5 | g |
| Methyl p-hydroxybenzoate, sodium | 0.4 | g | 0.4 | g | 0.4 | g |
| salt (preserving agent) | | | | | | |
| Conchyolin hydrolysate (MW: 600) at | 0.02 | g | | | | |
| 3% in water | | | | | | |
| Fragrance | 0.5 | g | 0.5 | g | | |
| Pure sodium hydroxide or citric acid | qs pH 5.3 | | qs pH 5.3 | | qs pH 5.3 | |
| monohydrate | | | | | | |
| Deionized water qs | 100 | g | 100 | g | 100 | g |

These compositions have a beautiful nacreous effect, are stable and have good cosmetic properties.

### EXAMPLES 4 and 5

Shampoos having the composition below were prepared:

| | Ex. 4 | | Ex. 5 | |
|---|---|---|---|---|
| Sodium lauryl ether sulfate (2.2 EO) as an | | | 15.5 | g AM |
| aqueous solution (Texapon AOS 225 UP from Cognis) | | | | |
| Sodium lauryl sulfate as an aqueous solution | 15.5 | g AM | | |
| (Texapon A 795 from Cognis) | | | | |
| Cocoyl amidopropyl betaine as an aqueous | 2.4 | g AM | | |
| solution (Tegobetaine F 50 from Goldschmidt) | | | | |
| Cocoyl betaine as an aqueous solution (Dehyton | | | 2.4 | g AM |
| AB 30 from Cognis) | | | | |
| Copolymer of acrylic or methacrylic acid esters, of | 0.6 | g AM | 0.6 | g AM |
| di(C1-4 alkyl)amino(C1-6 alkyl) methacrylate, of | | | | |
| PEG/PPG-30/5 allyl ether, of C10-30 PEG 20-25 alkyl | | | | |
| ether methacrylate and of C2-6 hydroxyalkyl | | | | |
| methacrylate crosslinked with ethylene glycol | | | | |
| dimethacrylate, as an emulsion at 20% by weight in water | | | | |
| (Carbopol Aqua CC Polymer from Noveon) | | | | |
| Hydroxypropyl guar trimethylammonium chloride | 0.1 | g | 0.1 | g |
| (Jaguar C13S from Rhodia) | | | | |
| Beta-cyclodextrin (cyclomaltoheptaose) | 1.72 | g AM | 1.72 | g AM |
| (Cavamax W7 Pharma) | | | | |
| Polydimethylsiloxane | 2.7 | g | 2.7 | g |
| (DC 200 Fluid 300 000 from Dow Corning) | | | | |
| Sodium chloride | 1.2 | g | 1.2 | g |
| Lactic acid | 0.28 | g | 0.28 | g |
| Preserving agents | qs | | qs | |
| Fragrance | 0.5 | g | 0.5 | g |
| Pure sodium hydroxide or citric acid monohydrate | qs pH 5.3 | | qs pH 5.3 | |
| Deionized water qs | 100 | g | 100 | g |

These compounds have a beautiful nacreous effect, are stable and have good cosmetic properties.

### EXAMPLE 6

A shampoo having the composition below was prepared:

| | Ex. 6 | |
|---|---|---|
| Sodium lauryl ether sulfate (2.2 EO) as an aqueous solution | 14 | g AM |
| (Texapon AOS 225 UP from Cognis) | | |
| Cocoyl betaine as an aqueous solution (Dehyton AB 30 from | 2 | g AM |
| Cognis) | | |
| Copolymer of acrylic or methacrylic acid esters, of di(C1-4 | 0.6 | g AM |
| alkyl)amino(C1-6 alkyl) methacrylate, of PEG/PPG-30/5 allyl ether, of | | |
| C10-30 PEG 20-25 alkyl ether methacrylate and of C2-6 hydroxyalkyl | | |
| methacrylate crosslinked with ethylene glycol dimethacrylate, as an | | |
| emulsion at 20% by weight in water (Carbopol Aqua CC Polymer | | |
| from Noveon) | | |
| Isopropyl myristate | 1.5 | g |
| Beta-cyclodextrin (cyclomaltoheptaose) | 1.72 | g AM |
| (Cavamax W7 Pharma) | | |
| Quaternized hydroxyethylcellulose (Polymer JR 400 from | 0.5 | g |
| Amerchol) | | |
| 2-Oleamido-1,3-octanediol | 0.01 | g |
| Sodium chloride | 1.2 | g |
| Lactic acid | 0.28 | g |
| Preserving agents | qs | |
| Fragrance | qs | |
| Pure sodium hydroxide or citric acid monohydrate | qs pH 5.3 | |
| Deionized water | qs 100 | g |

This composition has a beautiful nacreous effect, is stable and has good cosmetic properties.

### EXAMPLE 7

The following compositions were prepared:

| Amounts in table are given as % active | | |
|---|---|---|
| matter | A | B (invention) |
| Polyquaternium-10 (JR400 from | 0.4 | 0.4 |
| RHODIA) | | |
| Copolymer of de C10-C30 alkyl | 0.2 | — |
| PEG itaconate, C1-C4 | | |
| alkylaminoacrylate, and acrylic or | | |
| methacrylic acid (Structure Plus from | | |
| National Starch) | | |
| Copolymer of acrylic or methacrylic | — | 0.2 |
| acid esters, of di(C1-4 alkyl)amino(C1-6 | | |
| alkyl) methacrylate, of PEG/PPG-30/5 allyl | | |
| ether, of C10-30 PEG 20-25 alkyl ether | | |
| methacrylate and of C2-6 hydroxyalkyl | | |
| methacrylate crosslinked with ethylene | | |
| glycol dimethacrylate, as an emulsion at 20% | | |
| by weight in water (Carbopol Aqua CC | | |
| Polymer from LUBRIZOL | | |
| Isostearyl Neopentanoate | 0.5 | 0.5 |
| (Ceraphyl 375-ISP) | | |
| Beta-cyclodextrin | 2 | 2 |
| (cyclomaltoheptaose) | | |
| (Cavamax W7 Pharma-Wacker) | | |
| Sodium lauryl ether sulfate (2.2 EO) | 14 | 14 |
| (Texapon AOS 225 UP from Cognis) | | |
| Cocoamidopropyl betaine | 2.4 | 2.4 |
| Dimethicone (Mirasil DM 500000- | 1.5 | 1.5 |
| Rhodia) | | |
| Preservative | 0.45 | 0.45 |
| pH agent | Qs pH 6.8-7 | Qs pH 6.8-7 |
| Water | Qs 100% | Qs 100% |

6 g of composition A was applied on half-head of a human model, 6 g of composition B was applied on the other half-head of the same model. Compositions were rinsed then an expert evaluated the suppleness of hair. These operations were done on 10 models.

For 90% of persons, suppleness of hair treated with composition B was greater than the suppleness of hair treated with composition A.

What is claimed is:

1. A cosmetic composition comprising, in an aqueous medium:
(i) at least one cationic polymer, which is a product of polymerization of a monomer mixture comprising:
a) at least one vinyl monomer substituted with at least one amino group,
b) at least one hydrophobic nonionic vinyl monomer chosen from those of formulae (I) and (II):
CH₂═C(X)Z, and (I)
CH₂═CH—OC(O)R; (II)
in which:
X represents H or a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group; and
each R¹ independently is chosen from C₁-C₃₀ alkyl groups, hydroxylated C₂-C₃₀ alkyl groups, and halogenated C₁-C₃₀ alkyl groups;
c) at least one associative vinyl monomer,
d) at least one hydroxylated nonionic vinyl monomer, and
e) at least one semi-hydrophobic vinyl surfactant monomer;
(ii) at least one cyclodextrin or a derivative thereof; and
(iii) at least one surfactant.

2. The composition according to claim 1, wherein the at least one vinyl monomer substituted with at least one amino group is chosen from:
mono(C₁-C₄)alkylamino(C₁-C₈)alkyl (meth)acrylates,
di(C₁-C₄)alkylamino(C₁-C₈)alkyl (meth)acrylates, preferably di(C₁-C₄)alkylamino(C₁-C₆)alkyl (meth)acrylates,
mono(C₁-C₄)alkylamino(C₁-C₈)alkyl(meth)acrylamides,
di(C₁-C₄)alkylamino(C₁-C₈)alkyl(meth)acrylamides,
heterocyclic (meth)acrylamides comprising a nitrogen atom,
heterocyclic (meth)acrylates comprising a nitrogen atom,
and mixtures thereof.

3. The composition according to claim 2, wherein the at least one vinyl monomer substituted with at least one amino group is chosen from:
mono- or di(C₁-C₄ alkyl)amino(C₁-C₄alkyl) (meth)acrylates, mono- or di(C₁-C₄ alkyl)amino(C₁-C₄ alkyl)(meth)acrylamides, (meth)acrylamides or (meth)acrylates with a heterocyclic group comprising a nitrogen atom, and nitrogenous heterocycles containing vinyl group(s).

4. The composition according to claim 1, wherein the at least one vinyl monomer substituted with at least one amino group is present in an amount ranging from 10% to 70% by weight of the total weight of the monomer mixture.

5. The composition according to claim 1, wherein the at least one hydrophobic nonionic vinyl monomer is chosen from: C₁-C₃₀ alkyl (meth)acrylates, (C₁-C₃₀ alkyl)(meth)acrylamides, styrene, substituted styrenes, vinyl esters, unsaturated nitriles, and unsaturated silanes.

6. The composition according to claim 1, wherein the at least one hydrophobic nonionic vinyl monomer is present in an amount ranging from 20% to 80% by weight of the total weight of the monomer mixture.

7. The composition according to claim 1, wherein the at least one associative vinyl monomer is chosen from the compounds of formula (III): 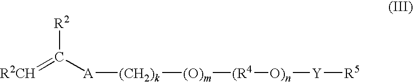
wherein:
each R² is independently chosen from a hydrogen atom, a methyl group, a —C(O)OH group, and a —C(O)OR³ group;
R³ is a C₁-C₃₀ alkyl;
A is chosen from —CH₂C(O)O—, —C(O)O—, —O—, CH₂O, —NHC(O)NH—, —C(O)NH—, —Ar—(CE₂)_{z}—NHC(O)O—, —Ar—(CE₂)ᵣ—NHC(O)NH—, and —CH₂CH₂—NHC(O)— groups;
Ar is a divalent aryl group;
E is chosen from a hydrogen atom and a methyl group;
z is an integer ranging from 0 to 1;
k is an integer ranging from 0 to 30;
m is an integer ranging from 0 to 1, with the proviso that when k is 0, m is 0, and when k is an integer ranging from 1 to 30, m is 1;
(R⁴—O)ₙ is a polyoxyalkylene, which is a homopolymer, a random copolymer or a block copolymer, with C₂-C₄ oxyalkylene units,
R⁴ is chosen from C₂H₄, C₃H₆, C₄H₈, and mixtures thereof,
n is an integer ranging from 5 to 250,
Y is chosen from —R⁴⁰—, —R⁴NH—, —C(O)—, —C(O)NH—, R⁴NHC(O)NH—, and —C(O)NHC(O)—; and
R⁵ is a substituted or unsubstituted alkyl chosen from: linear C₈-C₄₀ alkyls, branched C₈-C₄₀ alkyls, C₈-C₄₀ alicyclics, phenyls substituted with a C₂-C₄₀ alkyl group, C₂-C₄₀ alkyls substituted with an aryl group, and C₈-C₈₀ complex esters.

8. The composition according to claim 7, wherein the at least one associative vinyl monomer is chosen from: polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, polyethoxylated cholesteryl (meth)acrylates, and mixtures thereof,
wherein the polyethoxylated portion of the monomer comprises from 5 to 100 ethylene oxide units.

9. The composition according to claim 1, wherein the at least one associative vinyl monomer is present in an amount ranging from 0.001% to 25% by weight of the monomer mixture.

10. The composition according to claim 1, wherein the at least one semihydrophobic vinyl surfactant monomer is chosen from the compounds of formula (IV) or (V): 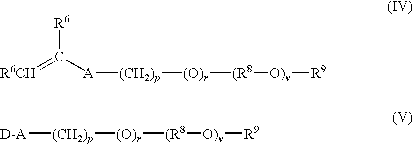
wherein:
each R⁶ independently is chosen from a hydrogen atom, a C₁-C₃₀ alkyl, —C(O)OH and C(O)OR⁷;
R⁷ is a C₁-C₃₀ alkyl;
A is chosen from —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}—NHC(O)NH— and —CH₂CH₂NHC(O)— groups;
Ar is a divalent aryl group;
E is chosen from a hydrogen atom and a methyl group;
z is an integer ranging from 0 or 1;
p is an integer ranging from 0 to 30;
r is an integer ranging from 0 or 1, with the proviso that when p is 0, r is 0, and when
p is an integer ranging from 1 to 30, r is 1;
(R⁸—O)ᵥ is a polyoxyalkylene which is a homopolymer, a random copolymer or a block copolymer with C₂-C₄ oxyalkylene units, in which R⁸ is chosen from C₂H₄, C₃H₆, C₄H₈ and mixtures thereof, and v is an integer ranging from 5 to 250;
R⁹ is chosen from a hydrogen atom and a C₁-C₄ alkyl; and
D is chosen from C₈-C₃₀ alkenyl and a C₈-C₃₀ alkenyl substituted with a carboxyl group.

11. The composition according to claim 1, wherein the at least one semi-hydrophobic vinyl surfactant monomer is chosen from the following formulae:
CH₂═CH—O(CH₂)ₐO(C₃H₆O)_{b}(C₂H₄O)_{c}H or
CH₂═CHCH₂O(C₃H₆O)_{d}(C₂H₄O)ₑH;
wherein:
a is an integer ranging from 2 to 4;
b is an integer ranging from 1 to 10;
c is an integer ranging from 5 to 50;
d is an integer ranging from 1 to 10; and
e is an integer ranging from 5 to 50.

12. The composition according to claim 11, wherein the at least one semi-hydrophobic vinyl surfactant monomer is present in an amount ranging from 0 to 25% by weight of the monomer mixture.

13. The composition according to claim 1, wherein the at least one hydroxylated nonionic vinyl monomer is chosen from: C₁-C₆ hydroxyalkyl (meth)acrylates and (C₁-C₄ hydroxyalkyl)(meth)acrylamides, and mixtures thereof.

14. The composition according to claim 1, wherein the at least one hydroxylated nonionic vinyl monomer is 2-hydroxyethyl methacrylate.

15. The composition according to claim 1, wherein the at least one hydroxylated nonionic vinyl monomer is present in an amount ranging from 0.01% to 10% by weight of the monomer mixture.

16. The composition according to claim 1, wherein the monomer mixture comprises, relative to the total weight of the monomer mixture:
a) from 20% to 60% by weight of the at least one vinyl monomer substituted with at least one amino group,
b) from 20% to 70% by weight of at least one hydrophobic nonionic vinyl monomer,
c)from 0.01% to 15% by weight of at least one associative vinyl monomer,
d) from 0.1% to 10% by weight of at least one semi-hydrophobic vinyl surfactant monomer,
e) from 0.01% to 10% by weight of at least one hydroxylated nonionic vinyl monomer,
f) from 0.001% to 5% by weight of at least one crosslinking monomer,
g) from 0.001% to 10% by weight of at least one chain-transfer agent, and
h) from 0 to 2% by weight of at least one polymeric stabilizer.

17. The composition according to claim 1, wherein the monomer mixture comprises:
a di(C₁-C₄ alkyl)amino(C₁-C₆ alkyl)methacrylate,
at least one C₁-C₃₀ alkyl ester of (meth)acrylic acid,
a C₁₀-C₃₀ alkyl methacrylate polyethoxylated with 20 to 30 mol of ethylene oxide,
a 30/5 polyethylene glycol/polypropylene glycol allyl ether,
a hydroxy(C₂-C₆ alkyl)methacrylate, and
an ethylene glycol dimethacrylate.

18. The composition according to claim 1, wherein the at least one cationic polymer (i) is present in an amount ranging from 0.01% to 10% by weight of the total weight of the composition.

19. The composition according to claim 1, wherein the at least one cyclodextrin is chosen from α-cyclodextrins, β-cyclodextrins and γ-cyclodextrins and the methyl derivatives thereof.

20. The composition according to claim 19, wherein the at least one cyclodextrin is chosen from β-cyclodextrins.

21. The composition according to claim 1, wherein the at least one cyclodextrin is present in an amount ranging from 0.2% to 30% by weight of the total weight of the composition.

22. The composition according to claim 1, wherein the at least one surfactant and the at least one cyclodextrin are present in amounts effective to form an insoluble complex in the composition.

23. The composition according to claim 1, wherein the at least one surfactant is anionic.

24. The composition according to claim 1, wherein the at least one surfactant is present in an amount ranging from 0.2% to 40% by weight relative to the total weight of the composition.

25. The composition according claim 1, further comprising at least one conditioning agent.

26. The composition according to claim 25, wherein the at least one conditioning agent is chosen from poly-α-olefins, fluoro oils, fluoro waxes, fluoro gums, carboxylic acid esters, silicones, cationic polymers other than the cationic polymers (i), mineral, plant or animal oils, ceramides and pseudoceramides, and mixtures thereof.

27. The composition according to claim 25, wherein the at least one conditioning agent is present in an amount ranging from 0.001% to 10% by weight of the total weight of the composition.

28. The composition according to claim 1, wherein the composition is a foaming detergent composition.

29. A cosmetic treatment process for keratin materials comprising: applying to the keratin materials,
in an aqueous medium:
(i) at least one cationic polymer, which is a product of polymerization of a monomer mixture comprising:
a) at least one vinyl monomer substituted with at least one amino group,
b) at least one hydrophobic nonionic vinyl monomer chosen from those of formulae (I) and (II):
CH₂═C(X)Z, and (I)
CH₂═CH—OC(O)R; (II)
in which:
X represents H or a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6,
each R independently is a C₁-C₃₀ alkyl group; and
each R¹ independently is chosen from C₁-C₃₀ alkyl groups, hydroxylated C₂-C₃₀ alkyl groups, and halogenated C₁-C₃₀ alkyl groups;
c) at least one associative vinyl monomer,
d) at least one hydroxylated nonionic vinyl monomer, and
e) one or more semi-hydrophobic vinyl surfactant monomers;
(ii) at least one cyclodextrin or a derivative thereof; and
(iii) at least one surfactant.

30. A cosmetic treatment process according to claim 29, wherein the application to the keratin materials is followed by rinsing the keratin material with water.

31. A method of using a composition as a shampoo, lotion, or conditioner, comprising: applying the composition to keratin materials, wherein the composition comprises:
in an aqueous medium:
(i) at least one cationic polymer, which is a product of polymerization of a monomer mixture comprising:
a) at least one vinyl monomer substituted with at least one amino group,
b) at least one hydrophobic nonionic vinyl monomer chosen from those of formulae (I) and (II):
CH₂═C(X)Z, and (I)
CH₂═CH—OC(O)R; (II)
in which:
X represents H or a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R independently is a C₁-C₃₀ alkyl group; and
each R¹ independently is chosen from C₁-C₃₀ alkyl groups, hydroxylated C₂-C₃₀ alkyl groups, and halogenated C₁-C₃₀ alkyl groups;
c) at least one associative vinyl monomer,
d) at least one hydroxylated nonionic vinyl monomer, and
e) one or more semi-hydrophobic vinyl surfactant monomers;
(ii) at least one cyclodextrin or a derivative thereof; and
(iii) at least one surfactant.
